(19) United States
(12) Patent Application Publication
(10) Pub. No.: US 2011/0293720 A1
(43) Pub. Date: Dec. 1, 2011

(54) PROGESTIN-CONTAINING DRUG DELIVERY SYSTEM

(75) Inventors: Sascha General, Berlin (DE); Ildikó Terebesi, Berlin (DE); Stefan Bracht, Glienicke Nordbahn (DE); Adrian Funke, Berlin (DE)

(73) Assignee: BAYER SCHERING PHARMA AKTIENGESELLSCHAFT, Berlin (DE)

(21) Appl. No.: 13/057,299

(22) PCT Filed: Aug. 7, 2009
(86) PCT No.: PCT/EP2009/060298

§ 371 Date: Aug. 10, 2011

Related U.S. Application Data
(60) Provisional application No. 61/087,334, filed on Aug. 8, 2008.

(30) Foreign Application Priority Data
Aug. 8, 2008 (EP) 08162105.4
Feb. 10, 2009 (EP) PCT/EP2009/000904

(51) Int. Cl. A61K 9/00; A61P 15/18; A61K 31/58

(52) U.S. Cl. 424/485; 424/484; 514/173; 424/486; 424/488

(57) ABSTRACT

The present invention relates to drug delivery compositions in the form of thin water-soluble films (wafers), which contain small particles that comprise at least one progestin and at least one protective agent. The protective agent provides effective taste-masking of the progestin due to limited release of the progestin in the mouth. The progestin is hence not absorbed via the buccal route, but rather via the enteral (per-oral) route.

### FIELD OF THE INVENTION

The present invention relates to drug delivery compositions in the form of thin water-soluble films (wafers), which contain particles that comprise at least one progestin and at least one protective agent. The protective agent provides effective taste-masking of the progestin due to limited release of the progestin in the mouth. The progestin is hence not absorbed via the buccal route, but rather via the enteral (per-oral) route. Thus, the wafer provided by the present invention can easily be modified to a unit dosage form which is essentially bioequivalent to a corresponding standard immediate-release (IR) oral tablet or capsule.

### BACKGROUND OF THE INVENTION

While drugs, such as progestins and/or estrogens, may be included in traditional standard oral tablet or capsule formulations to provide an accurate and consistent dose, such delivery forms have several disadvantages in both the administration and preparation of the drug. For example, it has been estimated that about 50% of the population have problems swallowing tablets (see Seager in J. Pharmacol. Pharm. 1998; 50; 375-382), and patients such as children or the elderly who will not, or cannot, swallow tablets or capsules represent a challenge for the pharmaceutical industry. The pharmaceutical industry has tried to meet this challenge by developing a number of different drug delivery systems, including rapid in-mouth disintegrating tablets, tablets which disintegrate in liquid prior to ingestion, liquids and syrups, gums and even transdermal patches. However, each of these drug delivery systems can pose their own problems.

Transdermal patches can be inconvenient and uncomfortable as well as rather expensive to produce. Furthermore, the drug flux through the skin can also raise very complex dosing issues. Liquids are particularly useful for children. However, liquids can be inconvenient for adults and can be relatively expensive to formulate, package and transport. Tablets that can be dissolved in a liquid before ingestion can also be useful. However, they can also be quite inconvenient in that they require liquid and a drinking container to be provided. Furthermore, time is required for disintegration and/or dissolution, even when effervescent tablets are used. Finally, these drug delivery systems can be quite messy as they typically leave a particulate and/or scum in the glass. Rapid in-mouth disintegrating tablets, such as chewable or self disintegrating tablets offer great convenience. However, chewable or self-disintegrating tablets often present real taste masking problems as the act of chewing can disrupt protective coatings. Furthermore, chewable or self-disintegrating tablets are often associated with an unpleasant mouthfeel. Moreover, the fear of swallowing, chewing, or choking on such solid shaped articles is still a concern in certain populations. In addition, the fragility/friability of such porous, and low-pressure moulded tablets makes them difficult to carry, store, handle and administer to patients, especially the children and the elderly.

Thus, there is a need for reliable delivery systems with improved patient compliance, i.e. where the dosing is easy and allows the patients to take their medications discretely wherever and whenever needed. Water-soluble films (wafers) provide many advantages compared to the above-mentioned drug delivery systems. Usually, such wafers dissolve quickly in the saliva present in the mouth thereby releasing the active ingredient(s) which, in turn, can then at least in part be absorbed via the buccal route and hence reduce or even avoid metabolisation by the liver (“first pass metabolism”). While such wafers in many instances represent an interesting alternative to the above-mentioned drug delivery systems there are certain situations where fast dissolution of the drug substance in the mouth (and hence buccal administration) is not necessarily desired.

For example, many active ingredients have an unpleasant taste, e.g. a bitter taste like the synthetic hormone drospirenone. When such active ingredients are quickly dissolved from the wafer, this may lead to a product which is unacceptable for the patient due to the unpleasant taste. Thus, taste-masking of such active ingredients represents a challenge. Furthermore, compared to an already approved and marketed oral tablet or capsule, buccal administration, by means of a wafer, would require adjustment of doses. This, in turn, means that the regulatory authorities, in such situations, would typically require full clinical trials in order to establish safety and efficacy of such a modified product. Thus, in cases where a bioequivalent alternative to an already approved and marketed oral tablet or capsule is desired, it may, however, still be desirable to take advantage of the wafer technology due to the many advantages this particular drug delivery system provides (no need for swallowing, chewing, etc.). However, the drug delivery system must necessarily be modified in such a way that absorption via the buccal route is avoided and it must be ensured that the active ingredient(s) is not effectively dissolved until it reaches the stomach or, optionally, the small intestine. As mentioned above, effective taste-masking is also an absolute requirement.

In summary, there is a need for drug delivery systems where the unpleasant taste of the active ingredient is effectively masked. In addition, or alternatively, there is a need for a drug delivery system which is bioequivalent to a standard IR oral tablet or capsule, but which, at the same time, do not possess the drawbacks of such a standard oral IR tablet or capsule.

The present inventor has provided a drug delivery system which, on the one hand, takes advantage of the attractive properties of wafers, but which, one the other hand, ensures that the unpleasant taste of the active ingredient(s) is effectively masked. This has been achieved by ensuring that once the wafer matrix is (quickly) dissolved in the saliva the progestin is, due to the presence of an appropriate protective agent, not dissolved in the mouth (and hence not administered via the buccal route), but is rather, by normal deglutition, transported to the stomach and/or the intestine where the progestin is effectively released. The drug delivery system of the invention is flexible in the sense that it may easily be adapted to a system which is bioequivalent to a standard IR oral tablet or capsule reference product.

Chewable taste-masked pharmaceutical compositions are described in U.S. Pat. No. 4,800,087.

Taste-masked orally disintegrating tablets (ODTs) are described in US 2006/0105038.

Taste-masking coating systems are described in WO 00/30617.

Taste-masked wafers are described in WO 03/030883.

Taste-masked powders and granules are described in EP 1 787 640.

Medicament-containing particles and solid preparations containing the particles are described in US 2007/0148230.

Non-mucoadhesive film dosage forms and techniques and methodologies for retarding the absorption of drugs from orally disintegrating films through the oral mucosa are described in WO 2008/040534. According to this document, mixing of donepezil with Eudragit® EPO results in immediate release characteristics of the active compound.

Solid dosage forms containing an edible alkaline agent as taste masking agent are described in WO 2007/109057.

Compositions and methods for mucosal delivery are described in WO 00/42992. This document further discloses dosage units wherein the active agent is encapsulated within a polymer.

Taste-masked pharmaceutical compositions prepared by coacervation are described in WO 2006/055142.

Compositions comprising sustained-release particles are described in U.S. Pat. No. 7,255,876.

WO 2007/074472 teaches that filler particles, e.g. having a particle size of >100 μm, give a coarse, gritty or sandy mouth feel when ingested as a mouth-dissolving tablet. Furthermore, this document discloses means to improve the mouth feel.

Xu et al., Int Pharm 2008; 359; 63 describe taste masking microspeheres for orally disintegrating tablets. However, the active agent is released relatively fast from these particles and complete taste masking is not achieved.

US 2007/0292479 describes film-shaped systems for transmucosal buccal application. Furthermore, the film-shaped systems described in US 2007/0292479 contain high amounts of cyclodextrin.

S I Pather, M J Rathbone and S Senel, Expert Opin. Drug Deliv 2008; 5; 531 review the current status and the future of buccal drug delivery systems and provide an insight into the difficulties and challenges in developing buccal dosage forms.

In the light of these prior art documents, the problems to be solved by the present invention include, but are not limited, to
formulate taste masked particles in such a size that they fit into drug delivery systems in the form of thin films (wafers);
formulate taste masked particles in such a way that they do not give any coarse, gritty or sandy mouth feel when released from the drug delivery systems into the mouth
uniformly incorporate taste masked particles into unit dosage forms in the form of thin films (wafers)
incorporate taste masked particles into thin water-soluble films comprising a water-soluble matrix polymer without dissolving or extracting said taste masked particles during manufacturing and/or storage

### SUMMARY OF THE INVENTION

In a first aspect, the present invention relates to a unit dosage form comprising a thin water-soluble film matrix, wherein
a) said film matrix comprises at least one water-soluble matrix polymer;
b) said film matrix comprises particles where said particles comprise at least one progestin and at least one protective agent, and where said particles have a d₉₀ particle size of 5.280 μm; and
c) said film matrix has a thickness of 5,300 μm.

Other aspects of the present invention will be apparent from the below description and the appended claims.

### DETAILED DESCRIPTION OF THE INVENTION

In the present context, the term “progestin” (also sometimes referred to as “gestagen” or “progestogen”) covers synthetic hormone compounds which are progesterone receptor agonists. The term is further meant to encompass all isomeric and physical forms of the progestins including hydrates, solvates, salts and complexes, such as complexes with cyclodextrins. Specific examples of progestins include, but is not limited to, progestins selected from the group consisting of levo-norgestrel, norgestrel, norethindrone (norethisterone), dienogest, norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desogestrel, 3-keto-desogestrel, norgestimate, gestodene, tibolone, cyproterone acetate, dienogest and drospirenone. Preferred progestins are gestodene, dienogest and drospirenone, in particular drospirenone. As discussed infra, the progestin may be complexed with a cyclodextrin.

The term “estrogen” is meant to encompass all compounds (natural or synthetic, steroidal or non-steroidal compounds) exhibiting estrogenic activity. Such compounds encompass inter alia conjugated estrogens, and phytoestrogens. The term is further meant to encompass all isomeric and physical forms of the estrogens including hydrates, solvates, salts and complexes, such as complexes with cyclodextrins. More particularly, the estrogen may be selected from the group consisting of ethinylestradiol, estradiol including therapeutically acceptable derivates (including esters) of estradiol, estrone, mestranol, estriol, estriol succinate and conjugated estrogens, including conjugated equine estrogens such as estrone sulfate, 17β-estradiol sulfate, 17α-estradiol sulfate, equilin sulfate, 17β-dihydroequilin sulfate, 17α-dihydroequilin sulfate, equilenin sulfate, 17β-dihydroequilenin sulfate and 17α-dihydroequilenin sulfate. Particular interesting estrogens are selected from the group consisting of ethinylestradiol, estradiol, estradiol sulfamates, estradiol valerate, estradiol benzoate, estrone, mestranol and estrone sulfate. More preferably, the estrogen is ethinylestradiol or estradiol. The most preferred estrogen is ethinylestradiol. As discussed infra, the estrogen may be complexed with a cyclodextrin.

When used herein, the term “therapeutically acceptable derivative of estradiol” refers to esters of estradiol; salts, such as sodium salts, of estradiol and estradio esters; as well as other derivatives known in the art. Typically, an ester of estradiol is in the 3-position or 7-position of estradiol. Specific examples of typical esters of estradiol include estradiol valerate, estradiol acetate, estradiol propionate, estradiol enantate, estradiol undecylate, estradiol benzoate, estradiol cypionate, estradiol sulfate, estradiol sulfamate, as well as salts thereof. Estradiol valerate is particularly preferred among the estradiol esters.

The term “estradiol” is intended to mean that the estradiol may be in the form of 17-α-estradiol or 17-β-estradiol. Preferably, the estradiol is in the form of 17-β-estradiol. The term “estradiol” also covers hydrated forms of estradiol, in particular estradiol hemihydrate.

The term “estrogen-cyclodextrin complex” or “estrogen complexed with cyclodextrin” is intended to mean a complex between an estrogen and a cyclodextrin, wherein the estrogen molecule is at least partially inserted into the cavity of a cyclodextrin molecule. The molar ratio between the estrogen and the cyclodextrin may be adjusted to any desirable value. In interesting embodiments of the invention, a molar ratio between the estrogen and the cyclodextrin is from about 2:1 to 1:10, preferably from about 1:1 to 1:5, most preferably from about 1:1 to 1:3, such as 1:1 or 1:2. Furthermore, the estrogen molecule may at least partially be inserted into the cavity of two or more cyclodextrin molecules, e.g. a single estrogen molecule may be inserted into two cyclodextrin molecules to give 1:2 ratio between estrogen and cyclodextrin. Similarly, the complex may contain more than one estrogen molecule at least partially inserted into a single cyclodextrin molecule, e.g. two estrogen molecules may be at least partially inserted into a single cyclodextrin molecule to give a 2:1 ratio between estrogen and cyclodextrin. Complexes between estrogens and cyclodextrins may be obtained by methods known in the art, e.g. as described in U.S. Pat. No. 5,798,338 and EP 1 353 700.

The term “ethinylestradiol-β-cyclodextrin complex” is intended to mean a complex, of any molar ratio, between ethinylestradiol and β-cyclodextrin. However, the ethinylestradiol-β-cyclodextrin complex is typically a complex between one molecule of ethinylestradiol and two molecules of β-cyclodextrin a 1:2 ethinylestradiol-β-cyclodextrin complex.

The term “progestin-cyclodextrin complex” or “progestin complexed with cyclodextrin” is intended to mean a complex between a progestin and a cyclodextrin, wherein the progestin molecule is at least partially inserted into the cavity of a cyciodextrin molecule. The molar ratio between the progestin and the cyclodextrin may be adjusted to any desirable value. In interesting embodiments of the invention, a molar ratio between the progestin and the cyclodextrin is from about 2:1 to 1:10, preferably from about 1:1 to 1:5, most preferably from about 1:1 to 1:3. Furthermore, the progestin molecule may at least partially be inserted into the cavity, of two or more cyclodextrin molecules, e.g. a single progestin molecule may be inserted into two cyclodextrin molecules to give 1:2 ratio between progestin and cyclodextrin. Similarly, the complex may contain more than one progestin molecule at least partially inserted into a single cyclodextrin molecule, e.g. two progestin molecules may be at least partially inserted into a single cyclodextrin molecule to give a 2:1 ratio between estrogen and cyclodextrin. Complexes between progestins and cyclodextrins may be obtained by methods known in the art, e.g. as described in U.S. Pat. No. 6,610,670 and references therein.

The term “drospirenone-β-cyclodextrin complex” is intended to mean a complex, of any molar ratio, between drospirenone and β-cyclodextrin as described in U.S. Pat. No. 6,610,670. However, the drospirenone-β-cyclodextrin complex is typically a complex between one molecule of drospirenone and three molecules of β-cyclodextrin, i.e. a 1:3 drospirenone-β-cyclodextrin complex.

The term “cyclodextrin” is intended to mean a cyclodextrin or a derivative thereof as well as mixtures of various cyclodextrins, mixtures of various derivatives of cyclodextrins and mixtures of various cyclodextrins and their derivatives. The cyclodextrin may be selected from the group consisting of α-cyclodextrin, β-cyclodextrin, γ-cyclodextrin and derivatives thereof. The cyclodextrin may be modified such that some or all of the primary or secondary hydroxyl groups of the macrocycle are alkylated or acylated. Methods of modifying these hydroxyl groups are well known to the person skilled in the art and many such modified cyclodextrins are commercially available. Thus, some or all of the hydroxyl groups of the cyclodextrin may have been substituted with an O—R group or an O—C(O)—R group, wherein R is an optionally substituted C₁₋₆-alkyl, an optionally substituted C₂₋₆-alkenyl, an optionally substituted C₂₋₆-alkynyl, an optionally substituted aryl or heteroaryl group. Thus, R may be a methyl, an ethyl, a propyl, a butyl, a pentyl, or a hexyl group, i.e. O—C(O)—R may be an acetate. Furthermore, the hydroxyl groups may be per-benzylated, per-benzoylated, benzylated or benzoylated on just one face of the macrocycle, i.e. only 1, 2, 3, 4, 5 or 6 hydroxyl groups is/are benzylated or benzoylated. Naturally, the hydroxyl groups may also be per-alkylated or per-acylated, such as per-methylated or per-acetylated, alkylated or acylated, such as methylated or acetylated, on just one face of the macrocycle, i.e. only 1, 2, 3, 4, 5 or 6 hydroxyl groups is/are alkylated or acylated, such as methylated or acetylated. Commonly used cyclodextrins are hydroxypropyl-β-cyclodextrin, DIMEB, RAMEB and sulfoalkyl ether cyclodextrins, such as sulfobutyl ether cyclodextrin (available under the trademark Captisol®). Although cyclodextrin-complexed active ingredients are indeed contemplated, the composition, in one embodiment of the invention, does not contain any cyclodextrin.

In the present context, the term. “C₁₋₆-alkyl” is intended to mean a linear or branched saturated hydrocarbon chain having from one to six carbon atoms, such as methyl; ethyl; propyl, such as n-propyl and isopropyl; butyl, such as n-butyl, isobutyl, sec-butyl and tert-butyl; pentyl, such as n-pentyl, isopentyl and neopentyl; and hexyl, such as n-hexyl and isohexyl. Likewise, the term “C₁₋₄-alkyl” is intended to mean a linear or branched saturated hydrocarbon chain having from one to four carbon atoms, such as methyl; ethyl; propyl, such as n-propyl and isopropyl; and butyl, such as n-butyl, isobutyl, sec-butyl and tert-butyl.

Although various cyclodextrin complexes of progestins and estrogens are described above, it is currently preferred that neither the progestin, nor the estrogen, is, complexed with a cyclodextrin. Accordingly, in a preferred embodiment, the unit dosage form of the invention does not contain a cyclodextrin.

As indicated above, the particles containing the progestin should be prepared in such a way that as little progestin as possible is released in the mouth, while as much progestin as possible is released in the stomach or, optionally, in the small intestine. This can be achieved by combining the progestin with a protective agent as will be discussed infra.

As will be known by the person skilled in the art, the typical residence time of disintegrating dosage forms in the mouth is typically below 3 minutes. In case (micro)particles are released from such dosage forms in the mouth, the same applies to these (micro)particles. Thus, the typical residence time of these (micro)particles in the mouth is about 3 minutes (this is meant to include the time from intake until the disintegration of the dosage form). Consequently, effective taste-masking may be investigated by in vitro dissolution tests in small volumes of a liquid simulating the saliva, and it can reasonably be assumed that effective taste-masking is achieved when, in the early time points from 0 to 3 minutes, the drug substance in 10 ml of a dissolution medium (typically an aqueous solution of pH 6) is either not detected or the detected amount is below the threshold for identifying its taste. It is evident that the absolute threshold for identifying the taste of a drug substance is dependent on the nature and dose of the drug substance. In the case of drospirenone, said threshold is higher than about 25% (w/w) when drospirenone is applied at a dosage level of 3 mg.

Thus, in order to effectively mask the unpleasant taste of the progestin, the protective agent must ensure that no or only very limited amounts of the progestin is dissolved under conditions simulating the conditions prevailing in the mouth. More particularly, it is preferred that less than 25% (w/w), such as less than 20% (w/w), more preferably, less than 15% (w/w), such as less than 10% (w/w), most preferably less than 5% (w/w) of the progestin is dissolved from the unit dosage form within 3 minutes as determined in an in vitro dissolution experiment representing the conditions in the mouth. A suitable in vitro dissolution experiment is described in example 8A herein. Basically, the dosage form is placed onto the bottom of a glass beaker. Then, 10 ml of simulated saliva pH 6.0 (composition: 1.436 g disodium phosphate dihydrate, 7.98 g monopotassium phosphate, and 8.0 g sodium chloride are dissolved in 950 ml water, adjusted to pH 6.0 and made up to 1000 ml) at 37° C. as dissolution medium is added into the beaker. Typically, the experiment is performed without any stirring or shaking (except for a gentle shaking within the first five seconds of the experiment in order to safeguard complete wetting of the dosage form), provided that the dosage form is formulated in such a way that it disintegrates completely within 3 minutes applying this procedure. If the dosage form is not formulated in such a way, stirring or shaking may be applied in a way that ensures complete disintegration of the dosage form within 3 minutes. After 3 minutes, the content of the beaker is inspected visually, and a sample of the liquid is drawn, filtered and analyzed for the content of the drug substance.

In order to investigate and assess the taste-masking properties of the protected particles before incorporation in the unit dosage form of the invention, the dissolution test described in Xu et al., Intl J Pharm 2008; 359; 63 may be applied. In a preferred embodiment of the invention less than 20% (w/w), more preferably less than 15% (w/w), most preferably less than 10% (w/w) of the progestin is dissolved from the protected particles within 5 minutes as determined by a dissolution apparatus type II using distilled water at 37° C. as the dissolution media and 100 rpm as the stirring rate.

As indicated above, it is of utmost importance that the progestin is quickly and effectively released in the stomach and/or the intestine. As will be understood by the skilled person also this effect may be simulated by in vitro dissolution tests, and it can reasonably be assumed that effective release of the progestin in the stomach and/or the intestine is achieved if at least 70% (w/w), more preferably at least 80% (w/w), most preferably at least 90% (w/w) of the progestin is dissolved from the unit dosage form within 30 minutes as determined by United States Pharmacopoeia (USP) XXXI Paddle Method (apparatus 2) using 900-1000 ml of a suitable dissolution medium at 37° C. and 50-100 rpm, preferably either 50, 75 or 100 rpm, as the stirring rate. Alternatively, the unit dosage form may be assayed for a shorter period of time under similar conditions. In such cases, it is preferred that at least 70% (w/w), more preferably at least 80% (w/w), most preferably at least 90% (w/w) of the progestin is dissolved from the unit dosage form within 20 minutes, more preferably within 15 minutes, as determined by USP XXXI Paddle Method (apparatus 2) using 900-1000 ml a suitable dissolution medium at 37° C. as the dissolution media and 50-100 rpm, preferably either 50, 75 or 100 rpm, as the stirring rate.

A typical in vitro dissolution experiment is described in example 8B. The suitable dissolution medium may be selected so that it reflects physiological conditions in the stomach and/or the intestine and specific properties of the unit dosage form. Thus, a suitable dissolution medium may be selected from e.g. water, aqueous buffer solutions of pH 1-8 (such as pH 1.0, 1.2, 1, 3, 2.0, 4.5, 6.0 and 6.8), aqueous buffer solutions of pH 1-8 (such as pH 1.0, 1.2, 1.3, 2.0, 4.5, 6.0 and 6.8) with the addition of 0.1-3% (w/v) sodium dodecyl sulphate, simulated gastric fluid, simulated intestinal fluid (fasted or fed state).

In one embodiment, the suitable dissolution medium is selected from 900-1000 ml 0.05 M phosphate buffer pH 6.0; 0.05 M phosphate buffer pH 6.0 with 0.5% (w/v) sodium dodecyl sulphate; and 0.05 M phosphate buffer pH 6.0 with 1% (w/v) sodium dodecyl sulphate. Most preferably, the suitable dissolution medium is 1000 ml 0.05 M phosphate buffer pH 6.0 with 0.5% (w/v) sodium dodecyl sulphate.

In another embodiment, the suitable dissolution medium is selected from 900 ml 0.05 M acetate buffer pH 4.5; 0.05 M acetate buffer pH 4.5 with 0.5% (w/v) sodium dodecyl sulphate; and 0.05 M acetate buffer pH 4.5 with 1% (w/v) sodium dodecyl sulphate. In a preferred embodiment, the suitable dissolution medium is 900 ml 0.05 M acetate buffer pH 4.5 with 0.5% (w/v) sodium dodecyl sulphate when the protective agent, is a wax, and 900 ml 0.05 M phosphate buffer pH 4.5 (without sodium dodecyl sulphate) when the protective agent is a cationic polymethacrylate.

The above-discussed dissolution tests are described in more detail in examples 8B, 8C and 8D herein.

Examples of simulated gastric fluids and simulated intestinal fluids are described in the USP XXXI. There are, however, other compositions of simulated body fluids known in the pharmaceutical literature. As mentioned supra, the exact composition of the dissolution medium should be selected in such a way that it reflects the physiological conditions in the stomach and/or the intestine and the specific properties of the unit dosage form.

A variety of materials, which are all well-known to the person skilled in the art, can be employed as the protective agent according to the present invention. Specific examples of such protective agents include cationic polymethacrylates and waxes.

In a preferred embodiment of the invention, the protective agent is a cationic polymethacrylate copolymer based on di-C₁₋₄-alkyl-amino-C₁₋₄-alkyl methacrylates and neutral methacrylic acid C₁₋₆-alkyl esters. In a more preferred embodiment of the invention, the cationic polymethacrylate is a copolymer based on dimethylaminoethyl methacrylate and neutral methacrylic acid C₁₋₄-alkyl esters, such as a copolymer based on dimethyl-aminoethyl methacrylate, methacrylic acid methyl ester and methacrylic acid butyl ester. A particular preferred cationic polymethacrylate is poly(butyl methacrylate, (2-dimethyl aminoethyl) methacrylate, methyl methacrylate) 1:2:1. The cationic polymethacrylates mentioned above typically have an average molecular mass in the range of from 100,000 to 500,000 Da, such as an average molecular mass in the range of from 100,000 to 300,000 Da, e.g. an average molecular mass in the range of from 100,000 to 250,000 Da, preferably an average molecular mass in the range of from 100,000 to 200,000 such as an average molecular mass in the range of from 125,000 to 175,000 Da, e.g. an average molecular mass of about 150,000 Da.

Such cationic polymethacrylates are available from Degussa, Germany under the trade name Eudragit® E In particular Eudragit® E 100 is preferred.

In another preferred embodiment of the invention, the protective agent is a wax Examples of waxes include animal waxes, such as beewax, chinese wax, shellac wax, spermaceti wax and wool wax; vegetable waxes, such as carnauba wax, bayberry wax, candelilla wax, castor wax, esparto wax, ouricury wax, rice bran wax and soy wax; mineral waxes, such as ceresin wax, montan wax, ozocerite wax and peat wax; petroleum waxes, such as paraffin wax and microcrystalline wax; and synthetic waxes, such as polyethylene waxes, Fischer-Tropsch waxes, esterified and/or saponified waxes, substituted amide waxes and polymerised α-olefines. A particular preferred wax is carnauba wax.

The weight ratio between the progestin and the wax is typically in the range of from 1:1 to 1:4, such as about 1:1, about 1:2, about 1:3 or about 1:4.

As discussed above, the particles comprising the progestin and the protective agent should release as little progestin as possible in the mouth, while as much progestin as possible should be dissolved in the stomach and/or the intestine. This can be achieved, e.g., by embedding the progestin in the protective agent, for example in such a way that the progestin is present in a solid dispersion in the protective agent. This embodiment is particularly preferred when the protective agent is a cationic polymethacrylate.

Alternatively, the progestin may be coated with the protective agent. This embodiment is particularly preferred when the protective agent is a wax.

In the present context, the term “solid dispersion” is used in its commonly accepted meaning, i.e. as a dispersion, wherein the dispersed phase consists of amorphous particles or crystalline particles or individual molecules (molecular dispersion). Thus, when used herein, the term “solid dispersion” means any solid system in which a component A (such as a progestin) is dispersed at a level of small particles or even at the molecular level (molecular dispersion) within another component B (such as a protective agent).

In the present context, the term “molecularly dispersed” or “molecular dispersion” is used in its commonly accepted meaning, i.e. as a dispersion, wherein the dispersed phase consists of individual molecules. Thus, when used herein, the term “molecularly dispersed” or “molecular dispersion” means any solid, semi-solid or liquid system in which a component A (such as a progestin or an estrogen) is dispersed at the molecular level within another component B (such as a protective agent), so that component A neither can be detected in crystalline form by X-ray diffraction analysis, nor be detected in particulate form, by any microscopic technique. It should also be understood that component A is dissolved in component B regardless of the nature and physical state of B. Thus, the term “molecularly dispersed” may be used interchangeably with the term “molecularly dissolved”.

As can be seen from the examples provided herein, the particle size of the particles comprising the progestin and the protecting agent is, at least to a certain extent, dependent on the applied protective agent. When carnauba wax is used as the protective agent, the d₉₀ particle size measurement leads in some cases to unplausible high values which are attributed to the formation of secondary aggregates and agglomerates. Such aggregates and agglomerates are easily separated during the manufacturing of the wafers. The particle size values specified below refer to the primary particles and not to the particle size of aggregates and agglomerates.

As indicated above, the particles comprising the progestin and the protective agent have a d₉₀ particle size of ≦280 μm, such as ≦250 μm, e.g. ≦200 μm. In an interesting embodiment of the invention, the particles have a d₉₀ particle size of ≦175 μm, such as a d₉₀ particle size of ≦150 μm, e.g. a d₉₀ particle size of ≦100 μm.

Stated differently, the particles comprising the progestin and the protective agent typically have a d₉₀ particle size in the range of from 30-280 μm, such as in the range of from 40-250 μm, e.g. in the range of from 50-200 μm or in the range of from 50-150 μm. Specific examples of d₉₀ particle sizes include values of about 30 μm, about 40 μm, about 50 μm, about 60 μm, about 70 μm, about 80 μm, about 90 μm, about 100 μm, about 110 μm, about 120 μm, about 130 μm, about 140 μm, and about 150 μm. Analogously, the d₅₀ particle size is typically in the range of from 5-80 μm, more typically in the range of from 10-75 μm. Specific examples of d₅₀ particle sizes include values of about 5 μm, about 10 μm, about 15 μm, about 20 μm, about 30 μm, about 40 μm, about 50 μm, about 60 μm, about 70 μm, and about 80 μm.

When used herein, the term “d₉₀ particle size” is intended to mean that the particle size distribution is so that at least 90% of the particles have a particle diameter of less than the specified value, calculated from the volume distribution curve under the presumption of spherical particles. In a similar way, the term “d₅₀ particle size” is intended to mean that the particle size distribution is so that at least 50% of the particles have a particle diameter of less than the specified value, calculated from the volume distribution curve under the presumption of spherical particles.

Therefore, it is important to note that whenever the terms “particle size”, “particle size distribution”, “particle diameter”, “d₉₀”, “d₅₀”, etc., are used herein it should be understood that the specific values or ranges used in connection therewith are always meant to be determined from the volume distribution curve under the presumption of spherical particles. The particle size distribution may be determined by various techniques, e.g. laser diffraction, and will be known to the person skilled in the art. The particles may be spherical, substantially spherical, or non-spherical, such as irregularly shaped particles or ellipsoidally shaped particles. Ellipsoidally shaped particles or ellipsoids are desirable because of their ability to maintain uniformity in the film forming matrix as they tend to settle to a lesser degree as compared to spherical particles. The particle size distribution of the particles comprising the progestin and the protective agent, when incorporated in the wafer, may be determined by dissolving the film forming matrix, separation of the protected particles, and drying the protected particles. The particle size distribution of the resulting particles may be determined as described above, e.g. by laser diffraction. For example, a Sympatec Helos laser diffractometer with a Sympatec Rhodos module aerial dispersion system can be used (Focal length 125 mm, volume of airstream 2.5 m³/h, prepressure 2 bar, dispersion pressure 3-4 bar, optical concentration 0.8-20%, measurement time: 2 seconds, optical model: Fraunhofer under the assumption of spherical particles).

Concerning the particles comprising the progestin and the protective agent, these particles typically constitute less than 60% by weight of the unit dosage form, preferably less than 50% by weight of the unit dosage form, more preferably less than 40% by weight of the unit dosage form. As will be understood, the amount of particles comprising the progestin and the protective agent is dependent on the potency of the selected progestin. Accordingly, the particles comprising the progestin and the protective agent generally constitute 0.1-50% by weight of the unit dosage form, preferably 1-40%, such as 2-40%, e.g. 5-30% by weight of the unit dosage form. Specific values include about 12%, about 15%, about 20%, and about 30% by weight of the unit dosage form.

As will be understood the particles comprising the therapeutically active agent(s) and the protective agent may contain additional excipients. However, in a preferred embodiment of the invention the particles consist essentially of the therapeutically active agent(s), i.e. a progestin, an estrogen or a combination of a progestin and an estrogen, and the protective agent.

As will be understood from the examples provided herein, the encapsulation efficiency is high and typically above 80%, such as above 85%, e.g. above 90%. Thus, the encapsulation efficiency is typically in the range of from 80-100%, such as in the range of from 85-100%, e.g. in the range of from 90-100%. When used herein, the term “encapsulation efficiency” means the ratio of the amount of therapeutically active agent incorporated in the protected particles versus the amount of active agent used for manufacturing of the protected particles.

The term “water-soluble film matrix”, when used herein; refers to a thin film which comprises, or consists of, a water-soluble polymer, particles comprising at least one progestin and at least one protective agent, and optionally other auxiliary components dissolved or dispersed in the water-soluble polymer. As used herein, the term “water-soluble polymer” refers to a polymer that is at least partially soluble in water, and preferably fully or predominantly soluble in water, or absorbs water. Polymers that absorb water are often referred to as being “water-swellable polymers”. The materials useful for the present invention may be water-soluble or water-swellable at room temperature (about 20° C.) and other temperatures, such as temperatures exceeding room temperature. Moreover, the materials may be water-soluble or water-swellable at pressures less than atmospheric pressure. Desirably, the water-soluble polymers are water-soluble, or water-swellable having at least 20% by weight water uptake. Water-swellable polymers having 25% by weight, or more, water uptake, are also useful. The unit dosage forms of the present invention formed from such water-soluble polymers are desirably sufficiently water-soluble to be dissolvable upon contact with bodily fluids, in particular saliva.

The water-soluble matrix polymer (typically constituting the major part of the water-soluble film matrix) can be selected from the group consisting of a cellulosic material, a synthetic polymer, a gum, a protein, a starch, a glucan and mixtures thereof.

Examples of cellulosic materials suitable for the purposes described herein include carboxymethyl cellulose, methyl cellulose, ethyl cellulose, hydroxymethyl cellulose, hydroxyethyl cellulose, hydroxypropyl cellulose, hydroxymethylpropyl cellulose, hydroxypropylmethyl cellulose and combinations thereof, Particularly preferred cellulosic materials are hydroxypropylmethyl cellulose and hydroxy-propyl cellulose, in particular hydroxypropylmethyl cellulose.

Examples of synthetic polymers include polymers commonly used as immediate-release (IR) coatings for pharmaceuticals, such as the polyvinyl alcohol polyethylene glycol (PVA-PEG) copolymers, which are commercially available in different grades under the trademark Kollicoat® IR. Further examples of synthetic polymers include polyacrylic acid and polyacrylic acid derivatives. A further advantage of using the above-mentioned synthetic polymers, in particular a PVA-PEG copolymer, is that they provide a stabilising effect on the therapeutically active substances present in the unit dosage form by limiting the oxidative degradation of progestins and estrogens which are unsubstituted in the 6- and/or 7-position. This effect is particularly pronounced when the therapeutically active agent (typically the estrogen) is dispersed, in particular molecularly dispersed, in the film matrix. Such degradations are well known in the field and is typical a problem in connection with the shelf life of the final solid preparation (see, for example, T. Hurley et al. Steroids 2002; 67; 165-174 and Van D. Reif et al. Pharmaceutical Research 1987; 4; 54-58). The stabilising effect can be observed, in particular, for the following estrogens:
ethinylestradiol, estradiol including therapeutically acceptable derivates of estradiol, estrone, mestranol, estriol, estriol succinate and conjugated estrogens, including conjugated equine estrogens such as estrone sulfate, 17β-estradiol sulfate and 17α-estradiol sulfate; and the following progestins:
levo-norgestrel, norgestrel, norethindrone (norethisterone), dienogest, norethindrone (norethisterone) acetate, ethynodiol diacetate, norethynodrel, allylestrenol, lynestrenol, norgestrienone, ethisterone, promegestone, desogestrel, 3-keto-desogestrel, norgestirnate and gestodene.

Examples of water-soluble gums include gum arable, xanthan gum, tragacanth, acacia, carageenan, guar gum, locust bean gum, pectin, alginates and combinations thereof.

Useful water-soluble protein polymers include gelatine, zein, gluten, soy protein, soy protein isolate, whey protein, whey protein isolate, casein, Levin, collagen and combinations thereof.

Examples of useful starches include gelatinised, modified or unmodified starches. The source of the starches may vary and include pullulan, tapioca, rice, corn, potato, wheat and combinations thereof.

Additional water-soluble polymers, which may be used in accordance with the present invention, include dextrin, dextran and combinations thereof, as well as chitin, chitosin and combinations thereof, polydextrose and fructose oligomers.

The amount of progestin incorporated in the unit dosage form of the invention is, of course, also dependent on the potency of the selected progestin, but will generally be in the range of from 0.1-30% (w/w) calculated on the basis of the unit dosage form. Typically, the amount of progestin incorporated in the unit dosage form of the invention is 0.5-25% (w/w), such as 1-20% (w/w), preferably 1-15% (w/w), such as 2-10% (w/w), e.g. about 6% (w/w) or about 7.5% (w/w).

As discussed supra, the unit dosage form preferably contains drospirenone as the progestinic component. The unit dosage form then typically contains 0.25-5 mg drospirenone, such as 1-4 mg drospirenone, e.g. 2-4 mg drospirenone, preferably 2.5-3.5 mg drospirenone, most preferably about 3 mg drospirenone. As discussed supra, drospirenone may be complexed with a cyclodextrin.

While the preferred progestin is drospirenone, incorporation of other progestins is indeed also within the scope of the present invention. More particularly, the unit dosage form may comprise desogestrel in an amount from 0.05-0.5 mg, preferably from 0.075-0.25 mg, such as 0.1 mg, 0.125 mg or 0.15 mg; ethynodiol diacetate in an amount from 0.25-2 mg, preferably 0.75-1.5 mg, such as 1 mg; levo-norgestrel in an amount from 0.025-0.3 mg, preferably from 0.075-0.25 mg, such as 0.1 mg or 0.15 mg; norethindrone (norethisterone) in an amount from 0.2-1.5 mg, preferably 0.3-1.25 mg, such as 0.4 mg, 0.5 mg or 1 mg; norethindrone (norethisterone) acetate in an amount from 0.5-2 mg, preferably 1-1.5 mg, such as 1 mg or 1.5 mg; norgestrel in an amount from 0.1-1 mg, preferably from 0.25-0.75 mg, such as 0.3 mg or 0.5 mg; norgestimate in an amount from 0.1-0.5 mg, preferably 0.15-0.3 mg, such as 0.18 mg, 0.215 mg or 0.25 mg; cyproterone acetate in an amount from 0.5-3 mg, such as 1-2 mg, preferably 2 mg; dienogest in an amount from 0.25-4 mg, such as 1-4 mg, preferably 2-3 mg, more preferably 2 mg; gestodene in an amount from 0.01-0.1 mg, such as 0.025-0.1 mg, e.g. 0.05-0.1 mg, preferably from 0.06-0.075 mg, such as 0.060 mg or 0.075 mg; and tibolone in an amount from 2-3 mg, such as 2.5 mg. As indicated supra the most preferred progestins are gestodene, dienogest and drospirenone, in particular drospirenone.

In addition to the water-soluble matrix polymer and the particles comprising the progestin and the protective agent, the unit dosage form of the invention may include a variety of various auxiliary components, such as taste-masking agents; organoleptic agents, such as sweeteners, taste modifiers and flavours, anti- and de-foaming agents; plasticizing agents; surfactants; emulsifying agents; agents improving the wetting of the particles; thickening agents; binding agents; cooling agents; saliva-stimulating agents, such as menthol; antimicrobial agents; colorants; etc. In a preferred embodiment of the invention, the unit dosage form does not contain an absorption enhancer.

Suitable sweeteners include both natural and artificial sweeteners. Specific examples of suitable sweeteners include, e.g.:

a) water-soluble sweetening agents such as sugar alcohols, monosaccharides, disaccharides and polysaccharides such as maltit, xylit, mannit, sorbit, xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;
b) water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;
c) dipeptide-based sweeteners, such as L-aspartic acid derived sweeteners, such as L-aspartyl-L-phenylalanine methyl ester (aspartame), L-alpha-aspartyl-N-(2,2,4,4 5 tetramethyl-3-thietanyl)-D-alaninamide hydrate, methyl esters of L-aspartyl-L phenylglycerin and L-aspartyl-L-2,5, dihydrophenylglycine, L-aspartyl-2,5-dihydro-L phenylalanine, L-aspartyl-L-(1-cyclohexyen)-alanine, and the like;
d) water-soluble sweeteners derived from naturally occurring water-soluble sweeteners, such as a chlorinated derivatives of ordinary sugar (sucrose), known, for example, under the product description of Sucralose®; and
e) protein-based sweeteners such as thaurnatoccous danielli (Thaurnatin I and II).

In general, an effective amount of sweetener is utilised to provide the level of sweetness desired for a particular unit dosage form, and this amount will vary with the sweetener selected. This amount will normally be from about 0.01% to about 20% by weight, preferably from about 0.05% to about 10% by weight, of the unit dosage form. These amounts may be used to achieve a desired level of sweetness independent from the flavour level achieved from any optional flavour oils used.

Useful flavours (or flavouring agents) include natural and artificial flavours. These flavourings may be chosen from synthetic flavour oils and flavouring aromatics, and/or oils, oleo resins and extracts derived from plants, leaves, flowers, fruits and so forth, and combinations thereof. Non-limiting examples of flavour oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavours such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavourings can be used individually or in combination. Commonly used flavours include mints such as peppermint, artificial vanilla, cinnamon derivatives, and various fruit flavours, whether employed individually or in combination. Flavourings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavourings include, but are not limited to acetaldehyde (apple); benzaldehyde (cherry, almond); cinnamicaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavours); butyraldehyde (butter, cheese); valeraldehyde (butter, cheese); citronellal (modified, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2-dimethyloctanal (greenfruit); and 2-dodecenal (citrus, mandarin); cherry; grape; essential oils, like menthol; mixtures thereof; and the like.

The amount of flavouring employed is normally a matter of preference, subject to such factors as flavour type, individual flavour, and strength desired. The amount may be varied in order to obtain the result desired in the final product. Such variations are within the capabilities of those skilled in the art without the need for undue experimentation. In general, amounts from about 0.01% to about 10% by weight of the film matrix are employed.

As discussed above, the unit dosage form may also include one or more surfactants, one or more emulsifying agents and/or other agents which aid in improving the wetting of the particles. This is, particularly preferred when the film matrix comprises particles where said particles comprise an estrogen (in particular ethinylestradiol) and the protective agent is a wax (in particular carnauba wax).

Examples of surfactants include nonionic, anionic, cationic and amphoteric surfactants. In particular, nonionic surfactants are preferred.

Examples of nonionic surfactants include, but are not limited to, the following:
Reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. The PEG-hydrogenated castor oils, available under the trademark Cremophor®, are especially suitable, in particular Cremophor® S9 (polyoxyethylene-400-monostearate) and Cremophor® EL (polyoxyl 35 castor oil).
Polyoxyethylene sorbitan fatty acid esters, also known as polysorbates, mono- and tri-lauryl, palmityl, stearyl and oleyl esters of the type known and commercially available under the trademark Tween®, including the following products:
   Tween® 20 [polyoxyethylene(20)sorbitanmonolaurate]
   Tween® 40 [polyoxyethylene(20)sorbitanmonopalmitate]
   Tween® 60 [polyoxyethylene(20)sorbitanmonostearate]
   Tween® 65 [polyoxyethylene(20)sorbitantristearate]
   Tween® 80 [polyoxyethylene(20)sorbitanmonooleate]
   Tween® 81 [polyoxyethylene(5)sorbitanmonooleate]
   Tween® 85 [polyoxyethylene(20)sorbitantrioleate]

Although PEG itself does not function as a surfactant, a variety of PEG-fatty acid esters have useful surfactant properties. Among the PEG-fatty acid monoesters, esters of lauric acid, oleic acid and stearic acid are most useful.
Sorbitan fatty acid esters, also known as spans, such as sorbitan monolaurate (span 20), sorbitan monostearate (span 60) and sorbitan monooleate (span 80).
Polyoxyethylene fatty acid esters, e.g., polyoxyethylene stearic acid esters of the type known and commercially available under the trademark Myrj®.
Polyoxyethylene-polyoxypropylene co-polymers and block co-polymers, e.g., of the type known and commercially available under the trademark Pluronic®, Emkalyx® and Poloxamer®
Dioctylsulfosuccinate or di-[2-ethylhexyl]-succinate.
Phospholipids, in particular, lecithins. Suitable lecithins include, in particular, soybean lecithins.
PEG mono- and di-fatty add esters, such as PEG dicaprylate, also known and commercially available under the trademark Miglyol® 840, PEG dilaurate, PEG hydroxystearate, PEG isostearate, PEG laurate, PEG ricinoleate, and PEG stearate.
Polyoxyethylene alkyl ethers, such as those commercially available under the trademark Brij®, e.g., Brij® 92V and Brij® 35.
Fatty acid monoglycerides, e.g., glycerol monostearate and glycerol monolaurate.
Saccharose fatty acid esters.
Cyclodextrins.
Tocopherol esters, e.g., tocophery acetate and tocopheryl add succinate.
Succinate esters, e.g., dioctylsulfosuccinate or related compounds, such as di-[2-ethylhexyl]-succinate.

Examples of anionic surfactants include, but are not limited to, sulfosuccinates, phosphates, sulfates and sulfonates. Specific examples of anionic surfactants are sodium lauryl sulfate, ammonium lauryl sulfate, ammonium stearate, alpha olefin sulfonate, ammonium laureth sulfate, ammonium laureth ether sulfate, ammonium stearate, sodium laureth sulfate, sodium octyl sulfate, sodium sulfonate, sodium sulfosuccinimate, sodium tridecyl ether sulfate and triethanolamine lauryl sulfate.

The amount may be varied in order to obtain the result desired in the final product. Such variations are within the capabilities of those skilled in the art without the need for undue experimentation. In general, amounts from about 0.01% to about 10% by weight of the film matrix are employed, preferably from about 0.05% to 5% by weight of the film matrix are employed.

As discussed above, the unit dosage form may also include an anti-foaming and/or de-foaming agent, such as simethicone, which is a combination of a polymethylsiloxane and silicon dioxide. Simethicone acts as either an anti-foaming or de-foaming agent which reduces or eliminates air from the film composition. Anti-foaming agents will aid in preventing the introduction of air into the composition, while de-foaming agents will aid removing air from the composition.

The unit dosage form of the invention is most preferably in the form of a thin film, which dissolves fast mainly-due to the large surface area of the film, which wets quickly when exposed to the moist oral environment. Contrary to fast-dissolving tablets, which are usually soft, friable and/or brittle, the film is solid and strong, but still flexible and does not require special packaging. As indicated above, the film is thin and can be carried in the patient's pocket, wallet or pocket book.

The film may be applied under or on the tongue, to the upper palatine, to the inner cheeks or any oral mucosal tissue, of the female mammal. The film may be rectangular, oval, circular, or, if desired, a specific shape, cut to the shape of the tongue, the palatine or the inner cheeks, may be applied. The film is rapidly hydrated and will adhere onto the site of application where it then rapidly disintegrates.

Concerning the dimensions of the unit dosage form of the invention, the water soluble film forming matrix is formed into a dry film which has a thickness of ≦300 μm, preferably ≦250 μm, more preferably ≦200 μm, most preferably ≦150 μm, such as ≦120 μm, e.g. ≦100 μm. As will be understood from the discussion above concerning the particle size of the particles comprising the progestin and the protective agent, the particle size, and therefore also to a certain extent the thickness of the film matrix, is somewhat dependent on the actually chosen protective agent. It is generally preferred, however, that the thickness of the film matrix is in the range of from 10-150 μm, such as 20-125 μm, e.g. 30-100 μm. More preferably, the thickness of the film matrix is in the range of from 35-90 μm, in particular in the range of from 40-80 μm. Specific, and preferred, examples include thicknesses of about 30 μm, about 40 μm, about 50 μm, about 60 μm, about 70 μm, about 80 μm, about 90 μm, about 100 μm, about 110 μm or about 120 μm.

Accordingly, in some embodiments of the invention, the thickness of the film matrix is ≦300 μm and the particles comprising the progestin and the protective agent have a d₉₀ particle size of ≦250 μm; the thickness of the film matrix is ≦250 μm and the particles comprising the progestin and the protective agent have a d₉₀ particle size of ≦200 μm; the thickness of the film matrix is ≦200 μm and the particles comprising the progestin and the protective agent have a d₉₀ particle size of ≦175 μm; the thickness of the film matrix is 5.200 μm and the particles comprising the progestin and the protective agent have a d₉₀ particle size of 5.150 μm; the thickness of the film matrix is ≦150 μm and the particles comprising the progestin and the protective agent have a d₉₀ particle size of ≦100 μm; or the thickness of the film matrix is ≦120 μm and the particles comprising the progestin and the protective agent have a d₉₀ particle size of ≦100 μm.

The surface dimension (surface area) of the film matrix is typically in the range of from 2-10 cm², such as in the range of from 3-10 cm², e.g. in the range of from 3-9 cm², more preferably in the range of from 4-8 cm². Specific, and preferred, examples of the surface area include surface areas of about 4, 4.5, 5, 5.5, 6, 6.5, 7, 7.5 or 8 cm². Most preferably, the surface area is about 5, 5.5, 6, 6.5 or 7 cm².

The total weight of the film matrix will typically be in the range of from 5-200 m such as in the range of from 5-150 mg, e.g. in the range of from 10-100 mg. More preferably, the total weight of the film matrix is in the range of from 10-75 mg, such as in the range of from 10-50 mg. Specific, and preferred, examples of the weight of the film matrix include weights of about 15 mg, about 20 mg, about 25 mg, about 30 mg, about 35 mg, about 40 mg, about 45 mg or about 50 mg.

The unit dosage form may be prepared and adhered to a second layer, i.e. a support or backing layer (liner) from which it is removed prior to use, i.e. before being introduced into the oral cavity. Preferably, the support or backing material is not water-soluble and may preferably consist of polyethylene-terephthalate, or other suitable materials well known to the skilled person.

In one embodiment of the invention, the unit dosage form contains the progestin as the only therapeutically active agent. However, in an interesting embodiment of the invention, the unit dosage form further comprises an estrogen.

In one embodiment of the invention, the estrogen-like the progestin is incorporated in the unit dosage form in a way allowing the estrogen not to be absorbed via the buccal route, i.e. so that as little estrogen as possible is dissolved in the mouth, while as much estrogen as possible is dissolved in the stomach and/or the intestine. This may be achieved by combining the estrogen with a protective agent in a similar way as discussed supra in connection with the progestinic component.

In one particular embodiment of the invention, the estrogen is incorporated in the particles already containing the progestin, i.e. according to this embodiment of the invention, the particles comprising the at least one progestin and the at least one protective agent further comprises at least one estrogen. Accordingly, in another aspect, the present invention relates to a unit dosage form comprising a thin water-soluble film matrix, wherein
a) said film matrix comprises at least one water-soluble matrix polymer;
b) said film matrix comprises particles where said particles comprises at least one progestin, at least one estrogen and at least one protective agent, and where said particles have a d₉₀ particle size of ≦280 μm; and
c) said film matrix has a thickness of ≦300 μm.

In an alternative embodiment of the invention, the estrogen is incorporated in separate particles, i.e. in particles comprising the protective agent, but no progestin. Accordingly, in a further aspect, the present invention relates to a unit dosage form comprising a thin water-soluble film matrix, wherein
a) said film matrix comprises at least one water-soluble matrix polymer;
b) said film matrix comprises particles where said particles comprises at least one progestin and at least one protective agent, and where said particles have a d₉₀ particle size of ≦280 μm;
c) said film matrix comprises particles where said particles comprises at least one estrogen and at least one protective agent, and where said particles have a d₉₀ particle size of ≦280 μm; and
d) said film matrix has a thickness of ≦300 μm.

The estrogen may be selected from the group consisting of ethinylestradiol, estradiol including therapeutically acceptable derivates of estradiol, estrone, mestranol, estriol, estriol succinate and conjugated estrogens. More preferably, the estrogen is selected from the group consisting of ethinylestradiol, estradiol, estradiol sulfamates, estradiol valerate, estradiol benzoate, estrone, mestranol and estrone sulfate. In highly preferred embodiments of the invention, the estrogen is ethinylestradiol or estradiol, in particular ethinylestradiol.

When ethinylestradiol is present in the unit dosage form, the unit dosage form typically contains 0.01-0.05 mg ethinylestradiol, preferably 0.02-0.03 mg ethinylestradiol. Specific amounts of ethinylestradiol include about 0.01 mg, about 0.015 mg, about 0.020 mg, about 0.025 mg or about 0.030 mg. Most preferably the amount of ethinylestradiol is about 0.02 mg ethinylestradiol or about 0.03 mg ethinylestradiol. As discussed supra, ethinylestradiol may be complexed with a cyclodextrin. Thus, in one particular interesting embodiment of the invention, the unit dosage form comprises about 3 mg drospirenone and about 0.02 mg ethinyl-estradiol, where the ethinylestradiol is optionally complexed with a cyclodextrin. In another particular interesting embodiment of the invention, the unit dosage form comprises about 3 mg drospirenone and about 0.03 mg ethinylestradiol.

When estradiol is present in the unit dosage form, the unit dosage form typically contains 1-3 mg estradiol, such as about 1 mg estradiol, about 2 mg of estradiol, or about 3 mg estradiol. Most preferably, the unit dosage form contains about 1 mg estradiol. Thus, in a particular interesting embodiment of the invention, the unit dosage form comprises about 0.5, 1 or 2 mg drospirenone and about 1 mg estradiol.

It will be understood that, apart from the specific amounts of estrogen to be incorporated in the particles, all other statements made above concerning the particles comprising the progestin and the protective agent apply mutatis mutandis to the aspects and embodiments where such particles, independently of the presence or absence of progestin, contain at least one estrogen. In other words, all statements concerning protective agents, dissolution properties, water-soluble matrix polymers, etc. also apply to the estrogen-containing particles and, as will be; understood, this is independent of whether the particles contain a progestin as well as an estrogen or whether the particles contain an estrogen as the only therapeutically active agent.

As mentioned supra, it is preferred according to this embodiment of the invention that a surfactant is comprised in the film matrix if the protective agent is wax. The weight ratio between the estrogen and the wax is typically in the range of from 1:1 to 1:4, such as about 1:1, about 1:2, about 1:3 or about 1:4.

In another embodiment of the invention, the estrogen—in contrast to the progestin—is incorporated in the unit dosage form in a way allowing the estrogen to be absorbed via the buccal route, i.e. so that as much estrogen as possible is dissolved in the mouth and hence absorbed via the oralmucosal route. This may be achieved by dissolving the estrogen (without being associated with any protective agent) in the water-soluble matrix polymer. Thus, in a still further aspect, the present invention relates to a unit dosage form comprising a thin water-soluble film matrix, wherein
a) said film matrix comprises at least one water-soluble matrix polymer, wherein at least one estrogen is dispersed, preferably molecularly dispersed, in said water-soluble matrix polymer;
b) said film matrix comprises particles where said particles comprises at least one progestin and at least one protective agent, and where said particles have a d₉₀ particle site of ≦280 μm; and
c) said film matrix has a thickness of ≦300 μm.

The estrogen may be selected from the group consisting of ethinylestradiol, estradiol including therapeutically acceptable derivates of estradiol, estrone, mestranol, estriol, estriol succinate and conjugated estrogens. More preferably, the estrogen is selected from the group consisting of ethinylestradiol, estradiol, estradiol sulfamates, estradiol valerate, estradiol benzoate, estrone, mestranol and estrone sulfate. In highly preferred embodiments of the invention, the estrogen is ethinylestradiol or estradiol, in particular ethinylestradiol.

It will be understood that when the estrogen component is incorporated in the unit dosage form according to the above embodiment of the invention (buccal administration), the bioavailability of the estrogen will be increased compared to the embodiments of the invention where the estrogen is associated with a protective agent. This, in turn, has the consequence that significantly lower dosages of the estrogen than stated above may be used

Thus, if estradiol is incorporated in the unit dosage form according to this particular embodiment of the invention, the unit dosage form contains 5-1000 μg of estradiol, such as 10-750 μg of estradiol, e.g. 25-500 μg of estradiol. Typically, the unit dosage form comprises 10-200 μg of estradiol, such as 10-60 μg of estradiol or >60-200 μg of estradiol.

In a preferred embodiment the unit dosage form contains estradiol in an “ultra-low” amount, i.e. 10-60 μg of estradiol, such as 25-60 μg of estradiol, preferably 30-50 μg of estradiol, more preferably 40-50 μg of estradiol, e.g. about 40, 45, 46 or 50 μg of estradiol. Alternatively, the “ultra low” amount is 10-60 μg of estradiol, such as 10-50 μg of estradiol, preferably 20-40 μg of estradiol, more preferably 25-35 μg of estradiol, e.g. about 30 μg of estradiol.

The unit dosage form may also contain estradiol in a “very low” amount i.e. >60-200 μg of estradiol, such as 70-160 μg of estradiol, e.g 70-150 μg of estradiol, preferably 80-150 μg of estradiol, such as 80-120 μg of estradiol or 120-150 μg of estradiol. Specific estradiol doses include 80, 85, 90, 100, 115, 120, 130, 150 and 160 μg of estradiol:

The unit dosage form may also contain a “medium low” amount of estradiol, i.e. >200-500 μg of estradiol, such, as 250-300 μg of estradiol, e.g. 260-280 μg of estradiol, more preferably 265-275 μg of estradiol e.g. about 270 μg of estradiol.

In still another embodiment, the unit dosage form may contain a “low” amount of estradiol, i.e. a dose of >500-1000 μg of estradiol, such as >500-750 μg of estradiol.

Specific examples of doses of estradiol which may be incorporated in the unit dosage form include doses of about 10, 12.5, 15, 20, 30, 40, 45, 46, 50, 60, 70, 80, 85, 90, 100, 115, 120, 130, 150, 160, 180, 200 or 270 μg of estradiol.

The above-mentioned doses preferably correspond to the daily dose. It should be understood that the above-mentioned doses are indicated with respect to anhydrous estradiol. If a hydrate of estradiol, such as estradiol hemihydrate, or a pharmaceutically acceptable ester of estradiol, such as estradiol valerate, is employed it will be understood that a dose which is therapeutically equivalent to the stated dose of anhydrous estradiol should be used. It is routine for those skilled in the art to determine pharmacologically/therapeutically equivalent doses of such other forms when the effective dose of anhydrous estradiol is known.

If ethinylestradiol is incorporated in the unit dosage form according to this particular embodiment of the invention, the unit dosage form typically contains 10-20 μg of ethinylestradiol, such as about 15 or 20 μg of ethinylestradiol.

### Manufacture

The unit dosage form of the invention may be prepared by processes and methods as shown in the examples and as described in WO 2007/073911.

The protected particles are typically prepared by dissolving the protective agent in a suitable organic solvent after which the progestin is added. Depending on the selection of the protective agent, the protective agent is either deposited on the surface of progestin particles (e.g. in the case carnauba wax is used as protective agent), or the progestin is incorporated as solid dispersion into particles comprising the protective agent and the progestin (e.g. in the case a cationic polymethacrylate copolymer is used as protective agent).

After removal of the organic solvent the resulting microparticles are dried and optionally milled and sieved. The milling equipment is selected according to the properties of the particles and the desired particle size, e.g. rotor mills or air jet mills may be used. Alternatively, the progestin may be dissolved together with the protective agent and spray-dried at a suitable temperature, e.g. 30-50° C., e.g. at a temperature of about 35° C. Typically, the protected particles prepared by spray-drying had a d₅₀ particle size of about 5-50 μm.

The matrix polymer solution (coating solution) is typically prepared by adding the water-soluble matrix polymer to a suitable solvent, such as water or a mixture of an alcohol and water. As Mentioned supra, it is preferred, if the protected particles comprise an estrogen (in particular ethinylestradiol) and the protective agent is a wax (in particular carnauba wax) that a surfactant is added. As will be understood, the time and conditions needed to dissolve the water-soluble matrix polymer will depend on the polymer and the solvent used. Thus, in some cases the water-soluble matrix polymer may dissolve easily at room temperature and with only gentle stirring, while in other cases it will be necessary to apply heat and vigorous stirring to the system. In a typical embodiment, the mixture is stirred for 1-4 hours, preferably for about 2 hours, or until a solution is obtained. The solution is typically stirred at a temperature of 60-80° C., such as about 70° C. After cooling to room temperature, the protected particles are optionally dispersed in a small volume of solvent or solvent mixtures and then poured into the matrix polymer solution and mixed thoroughly. The final mixing step and the optional pre-dispersing step as well can be performed by any method known to the skilled person, e.g. by using a pestle and mortar, or by stirring with an appropriate stirrer, such as a propeller stirrer, or by high sheer mixing, or by using rotor-stator mixing devices, such as, ultra-turrax, and/or applying ultrasound. The resulting solution (coating solution) can be used for coating immediately or within a few days, preferably within one day. The various amounts of solvent, matrix polymer, etc. are adjusted to reach a solid content of the coating solution of about 5-50% by weight, preferably 10-40% by weight, in particular 20-40% by weight, such as about 25% by weight, about 30% by weight, about 33% by weight, about 35% by weight and about 40% by weight.

Other excipients, auxiliary components and/or active drug substances may be added during any of the above mentioned steps.

As discussed supra the unit dosage form of the invention may contain an estrogen, which is dispersed, preferably molecularly dispersed, in the water-soluble film matrix. In this case, the estrogen is dissolved in a suitable solvent, such as ethanol and/or propylene glycol. This solution can be added to the solvents used for the coating solution before addition of the water-soluble matrix polymer. Alternatively, the solution can also be added after the water-soluble matrix polymer is already dissolved. In this case, the solution can be added either before, together or after the addition of the protected particles, before the final mixing step is performed.

If needed, the coating solution is degassed before being spread out on a suitable support or backing layer (liner). Examples of suitable liners include polyethylene-terephthalate (PET) liners, such as Perlasic® LF75 (available from Peden Converting), Loparex® LF2000 (available from Loparex BV) and Scotchpack® 9742 (available from 3M Drug delivery Systems). In one embodiment of the invention, the coating solution is spread out with the aid of a spreading box onto a suitable liner and dried for 12-24 hours at room temperature. A thin opaque film is then produced, which is subsequently cut or punched into pieces of the desired size and shape. Alternatively, the coating solution is coated as a thin film onto a suitable liner and in-line dried using an automated coating and drying equipment (e.g. by Coatema Coating Machinery GmbH, Dormagen, Germany) using a drying temperature of 40-100° C. A thin opaque film is then produced, which is subsequently cut or punched into pieces of the desired size and shape.

### Therapeutic Use and Administration

As is evident from the disclosure herein, the unit dosage forms of the invention are suitable for inhibition of ovulation in a female mammal, i.e. for providing contraception in a female mammal.

In a further interesting embodiment, the present invention relates to a pharmaceutical preparation or kit consisting essentially of 21, 22, 23 or 24, in particular 21 or 24, individually removable unit dosage forms (wafers) placed in a packaging unit, and 7, 6, 5 or 4, in particular 7 or 4, individually removable unit dosage forms (wafers) which do not contain any therapeutically active agents. In another embodiment of the invention the pharmaceutical preparation or kit does not contain any placebo wafers, i.e. the invention relates to a pharmaceutical preparation or kit consisting essentially of 21, 22, 23 or 24, in particular 21 or 24, individually removable unit dosage forms (wafers) according to the invention placed in a packaging unit. The unit dosage forms (wafers) may be individually packed, e.g. in single pouches, in a multiple unit blister pack, or the unit dosage forms (wafers) may be packed together in e.g. a multiple unit dispenser.

The preparation (or kit) may be a one-phase preparation, i.e. a preparation wherein the amounts of the progestin and the estrogen remain constant for the entire 21-, 22-, 23- or 24-day period. Alternatively, amounts of either or both active agents (i.e. the progestin and the estrogen) may be varied over the 21-, 22-, 23- or 24-day period to generate a multiple-phase preparation, e.g. a two- or three-phase preparation, such as descried in, e.g., U.S. Pat. No. 4,621,079.

In another aspect, the present invention relates to a unit dosage form of the invention for treating, alleviating or preventing a physical condition in a female mammal caused by insufficient endogenous levels of estrogen, such as osteoporosis, headaches, nausea, depression, vasomotor symptoms, symptoms of urogenital atrophy, decrease in bone mineral density or increased risk or incidence of bone fracture. In a preferred embodiment of the invention, the female mammal to be treated according to the invention is a postmenopausal woman, in particular a non-hysterectomised postmenopausal woman.

In a further aspect, the present invention relates to a unit dosage form of the invention for simultaneous inhibition of ovulation in a female mammal, i.e. for providing contraception in a female mammal, and for treating, alleviating or preventing a physical condition in a female mammal, caused by insufficient endogenous levels of estrogen, such as osteoporosis, headaches, nausea, depression, vasomotor symptoms, symptoms of urogenital atrophy, decrease in bone mineral density or increased risk or incidence of bone fracture. The group of women who may, in particular, benefit from this treatment are women in the perimenopause (also sometimes termed the “Menopausal Transition”, cf. the North American Menopause Society: Menopause Practice: A Clinician's Guide, 3. Edition, 2007), who are in, need of hormone replacement therapy, but still need contraceptive protection. It is preferred, according to this embodiment of the invention, that wafers containing the therapeutically active agents are administered for 23 or 24 days, in particular 24 days, followed by administration of wafers which do not contain any therapeutically active agents for 5 or 4 days, in particular 4 days, through a 28 days administration cycle.

In still another aspect, the present invention relates to a unit dosage form of the invention for treating, alleviating or preventing acne.

In still another aspect, the present invention relates to a unit dosage form of the invention for treating; alleviating or preventing hypertension.

In yet another aspect, the present invention relates to a unit dosage form of the invention for treating, alleviating or preventing premenstrual syndrome (PMS) and/or premenstrual dysphoric disorders (PMDD).

### Further Embodiments

1. A unit dosage form comprising a thin water-soluble film matrix, wherein
a) said film matrix comprises at least one water-soluble matrix polymer;
b) said film matrix comprises particles where said particles comprises at least one progestin and at least one protective agent, and where said particles have a d₉₀ particle size of ≦280 μm; and
c) said film matrix has a thickness of ≦300 μm.
   2. The unit dosage form according to embodiment 1 wherein said progestin is embedded in said protective agent.
   3. The unit dosage form according to embodiment 2, wherein said progestin is present in a solid dispersion in said protective agent.
   4. The unit dosage form according to embodiment 1, wherein said progestin is coated with said protective agent.
   5. The unit dosage form according to any of the preceding embodiments, wherein said protective agent is a cationic polymethacrylate.
   6. The unit dosage form according to embodiment 5, wherein said cationic polymethacrylate is a copolymer based on di-C₁₋₄-alkyl-amino-C₁₋₄-alkyl methacrylates and neutral methacrylic acid C₁₋₅-alkyl esters.
   7. The unit dosage form according to embodiment 6, wherein said cationic polymethacrylate is a copolymer based on dimethylaminoethyl methacrylate and neutral methacrylic acid C₁₋₄-alkyl esters.
   8. The unit dosage form according to embodiment 7, wherein said cationic polymethacrylate is a copolymer based on dimethyl-aminoethyl methacrylate, methacrylic acid methyl ester and methacrylic acid butyl ester.
   9. The unit dosage form according to embodiment 8, wherein said cationic polymethacrylate is poly(butyl methacrylate, (2-dimethyl aminoethyl) methacrylate, methyl methacrylate) 1:2:1.
   10. The unit dosage form according to any of embodiments 1-4, wherein said protective agent is a wax.
   11. The unit dosage form according to embodiment 10, wherein said wax is carnauba wax.
   12. The unit dosage form according to any of the preceding embodiments, wherein said particles have a d₉₀ particle size of ≦250 μm, such as a d₉₀ particle size of ≦200 μm, preferably a d₉₀ particle size of ≦175 μm, such as a d₉₀ particle size of ≦150 μm, e.g. a d₉₀ particle size of ≦100 μm.
   13. The unit dosage form according to any of the preceding embodiments, wherein said particles have a d₉₀ particle size in the range of from 30-280 μm, such as in the range of from 40-250 μm, e.g. in the range of from 50-200 μm or in the range of from 50-150 μm.
   14. The unit dosage form according to any of the preceding embodiments, wherein said progestin is selected from the group consisting of levo-norgestrel, norgestrel, norethindrone (norethisterone), dienogest, norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desogestrel, 3-keto-desogestrel, norgestimate, gestodene, tibolone, cyproterone acetate, dienogest and drospirenone.
   15. The unit dosage form according to embodiment 14, wherein said progestin is selected from the group consisting of drospirenone., gestodene and dienogest.
   16. The unit dosage form according to embodiment 15, wherein said unit dosage form comprises 0.25-5 mg drospirenone, such as 1-4 mg drospirenone, e.g. 2-4 mg drospirenone, preferably 2.5-15 mg drospirenone, most preferably about 3 mg drospirenone.
   17. The unit dosage form according to any of the preceding embodiments, wherein said water-soluble matrix polymer is selected from the group consisting of a cellulosic material, a gum, a protein, a starch, a synthetic polymer, a glucan, and mixtures thereof.
   18. The unit dosage form according to embodiment 17, wherein said water-soluble matrix polymer is a cellulosic material.
   19. The unit dosage form according to, embodiment 18, wherein said cellulosic, material is selected from the group consisting of carboxymethyl cellulose, methyl cellulose, ethyl cellulose, hydroxymethyl cellulose, hydroxyethyl cellulose, hydroxypropyl cellulose, hydroxymethylpropyl cellulose and hydroxypropylmethyl cellulose.
   20. The unit dosage form according to embodiment 19, wherein said cellulosic material is hydroxypropylmethyl cellulose or hydroxypropyl cellulose, preferably hydroxypropylmethyl cellulose.
   21. The unit dosage form according to embodiment 7; wherein said water-soluble matrix polymer is a synthetic polymer.
   22. The unit dosage form according to embodiment 21, wherein said synthetic polymer is a polyvinyl alcohol polyethylene glycol (PVA-PEG) copolymer,
   23. The unit dosage form according to any of the preceding embodiments, wherein said film matrix has a thickness of ≦250 μm, preferably ≦200 μm, such as ≦150 μm, more preferably ≦120, such as ≦100 μm.
   24. The unit dosage form according to embodiment 23, wherein said film matrix has a thickness in the range of from 10-150 μm, such as 20-125 μm, e.g. 30-100 μm, preferably 35-90 μm, more preferably 40-80 μm.
   25. The unit dosage form according to any of the preceding embodiments, wherein said unit dosage form further comprises at least one estrogen.
   26. A unit dosage form comprising a thin water-soluble film matrix, wherein
a) said film matrix comprises at least one water-soluble matrix polymer;
b) said film matrix comprises particles where said particles comprises at least one progestin, at least one estrogen and at least one protective agent, and where said particles have a d₉₀ particle size of ≦280 μm; and
c) said film matrix has a thickness of ≦300 μm.
   27. A unit dosage form comprising a thin water-soluble film matrix, wherein
a) said film, matrix comprises at least one water-soluble matrix polymer;
b) said film matrix comprises particles where said particles comprises at least one progestin and at least one protective agent, and where said particles have a d₉₀ particle size of ≦280 μm;
c) said film matrix comprises particles where said particles comprises at least one estrogen and at least one protective agent, and where said particles have a d₉₀ particle size of ≦280 μm;
d) said film matrix has a thickness of ≦300 μm.
   28. A unit dosage form comprising a thin water-soluble film matrix, wherein
a) said film matrix comprises at least one water-soluble matrix polymer, wherein at least one estrogen is dispersed, preferably molecularly dispersed, in said water-soluble matrix polymer;
b) said film matrix comprises particles where said particles comprises at least one progestin and at least one protective agent, and where said particles have a d₉₀ particle size of ≦280 μm; and
c) said film matrix has a thickness of ≦300 μm.
   29. The unit dosage form according to any of embodiments 25-28, wherein said estrogen is selected from the group consisting of ethinylestradiol, estradiol including therapeutically acceptable derivates of estradiol, estrone, mestranol, estriol, estriol succinate and conjugated estrogens.
   30. The unit dosage form according to embodiment 29, wherein said estrogen is selected from the group consisting of ethinylestradiol, estradiol, estradiol sulfamates, estradiol valerate, estradiol benzoate, estrone, mestranol and estrone sulfate.
   31. The unit dosage form according to embodiment 30, wherein said estrogen is ethinylestradiol or estradiol.
   32. The unit dosage form according to embodiment 31, wherein said estrogen is ethinylestradiol.
   33. The unit dosage form according to embodiment 31, wherein said estrogen is estradiol.
   34. The unit dosage form according to any of embodiments 25-33, wherein said unit dosage form comprises at least one surfactant.
   35. The unit dosage form according, to any of embodiments 26-34, wherein said film matrix comprises at least one surfactant.
   36. The unit dosage form according to any of the preceding embodiments, wherein less than 25% (w/w), preferably less than 20% (w/w), more preferably less than 15% (w/w), most preferably less than 5% (w/w) of the progestin is dissolved from the unit dosage form within 3 minutes when the unit dosage form is placed into a beaker with 10 ml of simulated saliva pH 6.0 at 37° C. as dissolution medium.
   37. The unit dosage form according to any of the preceding embodiments for use as a medicament.
   38. A unit dosage form according to any of embodiments 25-36 for the inhibition of ovulation in a female mammal.
   39. A unit dosage form according to any of embodiments 25-36 for providing contraception in a female mammal.
   40. A method for the inhibition of ovulation in a female mammal, said method comprising administering a unit dosage form as defined in any of embodiments 25-36 to a female mammal in need thereof.
   41. A method for providing contraception in a female mammal, said method comprising administering a unit dosage form as defined in any of embodiments 25-36 to a female mammal in need thereof.
   42. A unit dosage form as defined in any of embodiments 25-36 for treating, alleviating or preventing a physical condition in a female mammal caused by insufficient endogenous levels of estrogen.
   43. The unit dosage form according to embodiment 42, wherein said physical condition is selected from the group consisting of osteoporosis, headaches, nausea, depression, vasomotor symptoms, symptoms of urogenital atrophy, decrease in bone mineral density, and increased risk or incidence of bone fracture.
   44. A method for treating, alleviating or preventing a physical condition in a female mammal caused by insufficient endogenous levels of estrogen, said method comprising administering a unit dosage form as defined in any of embodiments 25-36 to a female mammal in need thereof
   45. The method according to embodiment 44, wherein the physical condition is selected from the group consisting of osteoporosis, headaches, nausea, depression, vasomotor symptoms, symptoms of urogenital atrophy, decrease in bone mineral density, and increased risk or incidence of bone fracture.

The invention is further illustrated by the following non-limiting examples.

### EXAMPLES

### Example 1

### Preparation of Particles Comprising a Protective Agent

### Example 1A

### Drospirenone/Carnauba Wax

80 g of carnauba wax (Pharm. Grade) was dissolved in 1 kg of n-heptane at 60° C. in a 2 litre double-walled glass beaker while stirred at 400 rpm until a clear solution was obtained.

80 g of micronized (d₅₀=2.2 μm; d₉₀=4.8 μm) drospirenone was added slowly to the solution to avoid clumping while the stirring rate was adjusted to 600 rpm. The mixture was cooled to 20° C. at a cooling rate of 20° C./hour to yield the drug containing microparticles coated with Carnauba wax.

The drospirenone-containing microparticles were filtrated using a cellulose acetate filter membrane and a glass filter unit. The microparticles were subsequently washed with 300 ml ethanol (96%) to remove n-heptane residues and non-encapsulated drospirenone.

The filtered microparticles were transferred to a glass bowl and dried for 2 hours at 30° C.

Batches of the resulting protected particles, wherein the drospirenone is coated with the protective agent, had the below particle sizes. As can be seen, for some batches the measured d₉₀ particle size is high due to secondary agglomeration. The true d₉₀ particle size value of the primary particles is estimated to be between 40 and 60 μm.

| Batch No. | d₅₀ (μm) | d₇₀ (μm) | d₉₀ (μm) |
|---|---|---|---|
| 1 | 11.6 | 19 | 50 |
| 2 | 16.0 | 50 | 265 |
| 3 | 12.3 | 20 | 175 |
| 4 | 12.8 | 20 | 224 |

The encapsulation efficiency was greater than 90%

### Example 16

### Ethinylestradiol/Carnauba Wax

Ethinylestradiol-containing microparticles were prepared as described in example 1A using 80 g of micronized (d₅₀=1.5 μm; d₉₀=4.0 μm) ethinylestradiol instead of 80 g of drospirenone.

Batches of the resulting protected particles, wherein the ethinylestradiol is coated with the protective agent, had the below particle sizes. As can be seen, for some batches the measured d₉₀ particle size is high due to secondary agglomeration. The true d₉₀ particle size value of the primary particles is estimated to be between 30 and 75 μm.

| Batch No. | d₅₀ (μm) | d₇₀ (μm) | d₉₀ (μm) |
|---|---|---|---|
| 1 | 11.5 | 18 | 36 |
| 2 | 9.6 | 62 | 247 |
| 3 | 10.2 | 20 | 73 |

The encapsulation efficiency was greater than 90%.

### Example 1C

### Drospirenone/Eudragit® E 100 (Milling)

20 g of drospirenone and 80 g of Eudragit® E 100 were dissolved in 200 ml of a mixture of ethanol and acetone 7+23 (waw) in a 300 ml glass beaker while stirring at 200 rpm at room temperature for 1 hour. A clear solution was obtained.

The solution was then transferred into a siliconized pan. The solution was dried under ambient conditions in a hood for 3 days to remove the acetone. A sensual test was used to indicate the absence of acetone. The thus obtained stiff film had a thickness of a few millimeters and was manually broken into parts of about 10 cm². These parts were subsequently milled using a rotor mill (Retsch ultra centrifugation mill ZM200) under cooling with, dry ice. The milled product was sieved using a mesh of 100 μm. The resulting protected particles, wherein the drospirenone is present in a solid dispersion in the protective agent, had a d₅₀ particle size of 34 μm and a d₉₀ particle size of 100 μm. The protected particles are stored protected from heat (e.g. in a refrigerator) until further use. The encapsulation efficiency was greater than 90%.

### Example 1D

### Ethinylestradiol/Eudragit® E 100 (Milling)

Ethinylestradiol-containing microparticles were prepared as described in example 1C using 10 g of ethinylestradiol/90 g of Eudragit® E 100 instead of 20 g of drospirenone/80 g of Eudragit® E 100. The ethinylestradiol was found to be molecularly dispersed in a solid dispersion in the protective agent, as confirmed by X-ray analysis. The resulting protected particles, wherein the esthinylestradiol is present in molecularly dispersed form in the protective agent, had a d₅₀ particle size of 48 μm and a d₉₀ particle size of 136 μm. The protected particles are stored protected from heat (e.g. in a refrigerator) until further use. The encapsulation efficiency was greater than 90%

### Example 1E

### Ethinylestradiol/Eudragit® E 100 (Milling)

The experiment according to example 1D was repeated and the following particle size distribution was obtained: d₅₀ particle size=46 μm; d₉₀ particle size=122 μm. The encapsulation efficiency was greater than 90%

### Example 1F

### Drospirenone/Eudragit® E 100 (Milling)

The experiment according to example 1C was repeated and the following particle size distribution was obtained: d₅₀ particle size=40 μm; d₉₀ particle size=129 μm. The encapsulation efficiency was greater than 90%.

### Example 1G

### Drospirenone/Eudragit® E 100 (Spray-Drying)

20 g of drospirenone and 80 g of Eudragit® E 100 were dissolved in 1000 ml of ethanol (96%) and spray-dried with a laboratory spraydrier (Büchi 190, switzerland). The resulting protected particles, wherein the drospirenone is present in a solid dispersion in the protective agent, had a d₅₀ particle size of 6.6 μm and a d₉₀ particle size of 57 μm. The protected particles are stored protected from heat (e.g. in a refrigerator) until further use. The encapsulation efficiency was greater than 90%.

### Example 1H

### Ethinylestradiol/Eudragit® E 100 (Spray-Drying)

Ethinylestradiol-containing microparticles were prepared as described in example 1G using ethinylestradiol instead of drospirenone. The ethinylestradiol was found to be molecularly dispersed in a solid dispersion in the protective agent, as confirmed by X-ray analysis. The resulting protected particles, wherein the ethinylestradiol is present in molecularly dispersed form in the protective agent, had a d₅₀ particle size of 10 μm and a d₉₀ particle size of 73 μm. The protected particles are stored protected from heat (e.g. in a refrigerator) until further use The encapsulation efficiency was greater than 90%.

### Example 1I

### Ethinylestradiol/Eudragit® E 100 (Spray-Drying)

Ethinylestradiol-containing microparticles were prepared as described in example 1H using 10 g of ethinylestradiol/90 g of Eudragit® E 100 instead of 20 g of ethinylestradiol/80 g of Eudragit® E 100. The ethinylestradiol was found to be molecularly dispersed in a solid dispersion in the protective agent, as confirmed by X-ray analysis. The resulting protected particles, wherein the ethinylestradiol is present in molecularly dispersed form in the protective agent, had a d₅₀ particle size of 5.5 μm and a d₉₀ particle size of 13.8 μm. The protected particles are stored protected from heat (e.g. in a refrigerator) until further use. The encapsulation efficiency was greater than 90%.

### Example 2

### Preparation of Particle-Containing Film Matrix (Coating) Solutions

### Example 2A

### Kollicoat® IR Matrix/Drospirenone Particles/Ethinylestradiol Particles

43.96 g of Kollicoat® IR was, dissolved in 100 ml of purified water in a glass beaker at 60-80° C. while stirring at 100 rpm for 2 hours. A clear solution was obtained (polymer solution). After cooling, the evaporated water was replaced.

6 g of the particles prepared in example 1A (drospirenone) and 40 mg of the particles prepared in example 1B (ethinylestradiol) were slowly added to the polymer solution while stirring. The stirring speed and time were adjusted to obtain a homogenous dispersion (coating solution).

### Example 2B

### Kollicoat® IR Matrix/Drospirenone Particles/Ethinylestradiol Particles

A coating solution was prepared as described in example 2A except that after addition of the particles the mixture was homogenised by a high shear homogeniser.

### Example 2C

### Kollicoat® IR Matrix/Drospirenone Particles/Ethinylestradiol Particles

88.9 g of the particles prepared in example 1A (drospirenone) and 0.593 g of the particles prepared in example 1B (ethinylestradiol) were homogeneously dispersed in a mixture of 222 g purified water and 116 g ethanol 96% in a high shear homogenizer (Becomix RW 2.5). 1121 g of purified water was added and mixed with the particles dispersion. The particle dispersion was warmed to 60-80° C. 651 g of Kollicoat IR® was added and dissolved to obtain a polymer solution containing the homogeneously dispersed protected particles (coating solution). After cooling of the coating solution to room temperature is was degassed over night under vacuum.

### Example 2D

### Kollicoat® IR Matrix/Drospirenone Particles/Ethinylestradiol Particles

43.96 g of Kollicoat® IR was dissolved in 80 ml of purified water in a glass beaker at 60-80° C. while stirring at 10.0 rpm for 2 hours. A clear solution was obtained (polymer solution). After cooling, the evaporated water was replaced.

6 g of the particles prepared in example 1A (drospirenone) and 40 mg of the particles prepared in example 1B (ethinylestradiol) were dispersed in a mixture of 8 ml ethanol and 12 ml water and then added to the polymer solution while stirring. The stirring speed and time were adjusted to obtain a homogenous dispersion (coating solution).

### Example 2E

### Kollicoat® IR Matrix Containing Menthol/Drospirenone Particles/Ethinylestradiol Particles

42.96 g of Kollicoat® IR was dissolved in 77 ml of purified water in a glass beaker at 60-80° C. while stirring at 100 rpm for 2 hours. A clear solution was obtained (polymer solution). After cooling, the evaporated water was replaced.

1 g menthol was dissolved in 3 ml of ethanol (96%) with stirring under ambient conditions (ethanol solution).

6 g of the particles prepared in example 1A (drospirenone) and 40 mg of the particles prepared in example 1B (ethinylestradiol) were dispersed in a mixture of 8 ml ethanol and 12 ml water and then added to the polymer solution while stirring. The stirring speed and time were adjusted to obtain a homogenous dispersion. Subsequently, the ethanol solution was added (coating solution).

### Example 2F

### Kollicoat® IR Matrix/Ethinylestradiol/Drospirenone Particles

222 mg of ethinylestradiol was dissolved in 116.4 g of ethanol (96%) with stirring under ambient conditions in a high shear mixer (Becomix 2.5 RW). Subsequently, 222 g of purified water was added (ethanol/water solution).

89 g of the particles prepared in example 1A (drospirenone) were dispersed in the ethanol/water solution. Then, 1121 g of purified water was added, mixed with the dispersion and heated to 60-80° C. 652 g of Kollicoat® IR was added and dissolved to obtain a solution (coating solution).

### Example 2G

### Kollicoat® IR Matrix/Estradiol/Drospirenone Particles

88.9 g of the particles prepared in example 1A (drospirenone) were dispersed in 474 g of a 1:1 mixture of ethanol (96%) and purified water in a high shear mixer (Becomix 2.5 RW) at ambient temperature (dispersion).

1.39 g estradiol hemihydrate was dissolved in 46.3 g of ethanol (96%) with stirring under ambient conditions (ethanol solution). The ethanol solution was then added to the dispersion and homogenised. Subsequently, a mixture of 155.6 g of ethanol (96%) and 785 g of purified water was added drop-wise and homogenised. The mixture was then heated 60-80° C. 650 g of Kollicoat® IR was added and dissolved to obtain a solution (coating solution).

### Example 2H

### Kollicoat® IR Matrix/Estradiol Valerate/Drospirenone Particles

43.882 g of Kollicoat® IR was dissolved in 78 ml of purified water in a glass beaker at 60-80° C. while stirring at 100 rpm for 2 hours. A clear solution was obtained (polymer solution). After cooling, the evaporated water was replaced.

118 mg estradiol valerate was dissolved in 2 ml of ethanol (96%) with stirring under ambient conditions (ethanol solution).

6 g of the particles prepared in example 1A (drospirenone) were dispersed in a mixture of 8 ml ethanol and 12 ml water and then added to the polymer solution while stirring. The stirring speed and time were adjusted to obtain a homogenous dispersion (coating solution). Subsequently, the ethanol solution was added (coating solution).

### Example 21

### HPMC Matrix/Drospirenone Particles/Ethinylestradiol Particles

37.5 g sorbitol and 37.5 g propylene glycol were dissolved in 750 g of purified water in a high shear mixer (Becomix RW2.5). 150 g of the particles prepared in example 1C (drospirenone) and 2 g of the particles prepared in example 1D (ethinylestradiol) were slowly added while stirring and homogenised until a homogeneous particle dispersion was obtained. 273 g hydroxypropylmethyl cellulose (HPMC) was strewed onto the aqueous particle dispersion and dissolved under stirring and homogenization without any further heating for 2 hours (coating solution).

### Example 2J

### HPMC Matrix Containing Menthol/Drospirenone Particles/Ethinylestradiol Particles

3.75 g sorbitol is dissolved in 58 ml of purified water at 60-80° C. in a glass beaker. 26.3 g hydroxypropylmethyl cellulose (HPMC) is strewed onto the aqueous solution and dissolved under stirring without any further heating for 2 hours (polymer solution).

3.75 g propylene glycol and 1 g menthol are dissolved in 2 ml of ethanol (96%) with stirring under ambient conditions (ethanol solution).

15 g of the particles prepared in example 1C (drospirenone) and 200 mg of the particles prepared in example 1D (ethinylestradiol) are slowly added to the cooled (−20° C.) polymer solution while stirring. The stirring speed and time are adjusted to obtain a homogenous dispersion. Subsequently, the ethanol solution is added and mixed (coating solution).

### Example 2K

### HPMC Matrix/Ethinylestradiol/Drospirenone Particles

375 g hydroxypropylmethyl cellulose (HPMC) is dissolved in 900 g of purified water at 60-80° C. in a high shear mixer (Beomix RW 2.5). The solution was subsequently cooled to 25-45° C. (polymer solution). To avoid air bubbles, the polymer solution is degassed for 15-20 hours under vacuum.

181 mg ethinylestradiol are dissolved in 45 g propylene glycol with stirring under ambient conditions (propylene glycol solution).

186 g of the particles prepared in example 1C (drospirenone) are slowly added to the cooled (˜20° C.) polymer solution while mixing and homogenising. The mixing and homogenisation speed and time are adjusted to obtain a homogenous dispersion. Subsequently, the propylene glycol solution is added and mixed (coating solution).

### Example 2L

### HPMC Matrix/Estradiol/Drospirenone Particles

353 g hydroxypropylmethyl cellulose (HPMC) is dissolved in 850 g of purified water at 60-80° C. in a high shear mixer (Beomix RW 2.5). The solution was subsequently cooled to 25-45° C. (polymer solution). To avoid air bubbles, the polymer solution is degassed for 15-20 hours under vacuum.

1.1 g estradiol hemihydrate are dissolved in 42.5 g propylene glycol with stirring under ambient conditions (propylene glycol solution).

170 g of the particles prepared in example 1C (drospirenone) are slowly added to the cooled (˜20° C.) polymer solution while mixing and homogenising. The mixing and homogenising speed and time are adjusted to obtain a homogenous dispersion. Subsequently, the propylene glycol solution is added and mixed (coating solution).

### Example 2M

### HPMC Matrix/Estradiol Valerate/Drospirenone Particles

3.75 g sorbitol is dissolved in 58 ml of purified water at 60-80° C. in a glass beaker. 27.382 g hydroxypropylmethyl cellulose (HPMC) is strewed onto the aqueous solution and dissolved under stirring without any further heating for 2 hours (polymer solution).

3.75 g propylene glycol and 118 mg estradiol valerate are dissolved in 2 ml of ethanol (96%) with stirring under ambient conditions (ethanol solution).

15 g of the particles prepared in example 1C (drospirenone) are slowly added to the cooled (˜20° C.) polymer solution while stirring. The stirring speed and time are adjusted to obtain a homogenous dispersion. Subsequently, the ethanol solution is added (coating solution).

### Example 2N

### Kollicoat® IR Matrix/Drospirenone Particles/Ethinylestradiol Particles

88.9 g of the particles prepared in example 1A (drospirenone) and 0.593 g of the particles prepared in example 1B (ethinylestradiol) were homogeneously dispersed in a mixture of 460 g purified water containing 0.05% (w/w) Tween® 80 in a high shear homogenizer (Becomix RW 2.5). 1000 g of purified water containing 0.05% (w/w) Tween® 80 was added and mixed with the particles dispersion. The particle dispersion was warmed to 60-80° C. 651 g of Kollicoat IR® was added and dissolved to obtain a polymer solution containing the homogeneously dispersed protected particles (coating solution). After cooling of the coating solution to room temperature, is was degassed over night under vacuum.

### Example 3

### Preparation of Wafers

### Example 3A

The coating solution was degassed and spread out, with the aid of a casting knife, onto a polyethylene-terephthalate (PET) liner (Perlasic® LF75) and dried for 24 hours at room temperature. An opaque film with a thickness of about 70 μm was produced. Wafers with a content of 3 mg drospirenone were obtained by punching out samples of 7 cm² size.

### Example 3B

The coating solution was degassed and coated as a thin film onto a polyethylene-terephthalate (PET) liner (Perlasic® LF75) and in-line dried using an automated coating and drying equipment (Coatema Coating Machinery GmbH, Dormagen, Germany). A drying temperature of 70° C. was applied. An opaque film with a thickness of about 70 μm was produced. Wafers with a content of 3 mg drospirenone and a total weight of about 50 mg were obtained by punching out samples of 7 cm² size.

### Example 3C

The coating solution was degassed and coated as a thin film onto a polyethylene-terephthalate (PET) liner (Perlasic®LF75) and in-line dried using an automated coating and drying equipment (Coatema Coating Machinery GmbH, Dormagen, Germany). A drying temperature of 70° C. was applied. An opaque film with a thickness of about 90 μm was produced. Wafers with a content of 3 mg drospirenone and a total weight of about 50 mg were obtained by punching out samples of 5 cm² size.

### Example 3D

The coating solution was degassed and coated as a thin film onto a polyethylene-terephthalate (PET) liner (Perlasic®LF75) and in-line dried using an automated coating and drying equipment (Coatema Coating Machinery GmbH, Dormagen Germany). A drying temperature of 70° C. was applied. An opaque film with a thickness of about 70 μm was produced. Wafers with a content of 3 mg drospirenone and a total weight of about 35 mg were obtained by punching out samples of 5 cm² size.

### Example 4

### Preparation of Wafers Containing Polystyrene Standard Particles

3.75 g sorbitol and 3.75 g propylene glycol were dissolved in 60 ml of purified water at 60-80° C. in a glass beaker. 27.3 g hydroxypropylmethyl cellulose (HPMC) was strewed onto the aqueous solution and dissolved under stirring without any further heating for 2 hours. Four solutions were prepared.

3.5 g of four different standard polystyrene particles (obtained from Polymer Standard Services) with diameters of 10 μm, 20 μm, 40 μm, and 50 μm, respectively, were slowly added to the four solutions while stirring. The stirring speed and time were adjusted to obtain a homogenous dispersion (coating solution).

The coating solutions were spread out, with the aid of a casting knife, onto a polyethylene-terephthalate (PET) liner (Perlasic® LF75) and dried for 24 hours at room temperature. Four opaque films with a thickness of about 100 μm were produced, each film containing about 50% polystyrene standard particles of different diameters. The films were cut into samples of 5 cm² size.

A test panel consisting of five test persons assessed the sensory mouth feel of the wafers. The wafers were completely randomized and all wafers looked alike. The test persons were informed that the wafers did not contain any active compound, but did not receive any further information regarding the formulation and composition of the wafers. The score was from 1 (no sensation) to 5 (sandy and gritty mouth feel). The obtained results (mean values) are compiled below:

Polystyrene particle
diameter (μm)

| | 10 | 20 | 40 | 50 |
|---|---|---|---|---|
| Mean score | 1 | 1.4 | 1.6 | 2.8 |

From the above results it can be concluded that the particle size is of importance of the mouth feel of the resulting wafer. Evidently, the lower the diameter of the particles, the more improved mouth feel.

### Example 5

### Preparation of Wafers Containing Drospirenone and No Protective Agent

500 mg of hydroxypropylmethyl cellulose (HPMC) was strewed onto 2 ml of purified water and dissolved under stirring at 60-80° C. for 2 hours.

30 mg micronized drospirenone was slowly added to the solution while stirring at 200 rpm for 1 hour at room temperature. A homogenous dispersion (coating solution) was obtained.

The coating solution was formed into opaque wafers as described in example 3A.

### Example 6

### Taste Evaluation

A taste panel assessed the bitterness (drospirenone has a bitter taste) of the wafers prepared from coating solutions as described in examples 2A, 2E, 2I, and example 5 (unprotected drospirenone). All wafers were manufactured as described in example 3A. The wafers were completely randomized and all wafers looked alike. The test persons were informed about the active drug substances present in the wafers and the dose, but did not receive any information about the specific formulation of the wafers. The test persons were advised to place the wafers onto the tongue and allow for disintegration without swallowing for three minutes. After that the test persons had to disgorge any remaining material from the mouth and then rinse the mouth with water.

The wafer prepared according to example 5 had a bitter taste. No bitter taste could be detected for, any of the other wafers.

Furthermore, the test persons were asked to describe the sensory mouth feel of the samples. All wafer formulations were rated acceptable.

### Example 7

### Formulations

### Example 7A

| Ingredient | Amount | | Function |
|---|---|---|---|
| Ethinylestradiol | 0.020 | mg | Active ingredient |
| Drospirenone | 3.0 | mg | Active ingredient |
| Eudragit ® E 100 | 12.18 | mg | Protective agent |
| HPMC | 27.3 | mg | Matrix polymer |
| Propylene glycol | 3.75 | mg | Softening agent |
| Sorbitol | 3.75 | mg | Sweetener |
| Total | 50 | mg | |

### Example 7B

| Ingredient | Amount | | Function |
|---|---|---|---|
| Ethinylestradiol | 0.020 | mg | Active ingredient |
| Drospirenone | 3.0 | mg | Active ingredient |
| Eudragit ® E 100 | 12.18 | mg | Protective agent |
| HPMC | 34.8 | mg | Matrix polymer |
| Total | 50 | mg | |

### Example 7C

| Ingredient | Amount | | Function |
|---|---|---|---|
| Ethinylestradiol | 0.020 | mg | Active ingredient |
| Drospirenone | 3.0 | mg | Active ingredient |
| Eudragit ® E 100 | 12.18 | mg | Protective agent |
| Kollicoat ® IR | 34.8 | mg | Matrix polymer |
| Total | 50 | mg | |

### Example 7D

| Ingredient | Amount | | Function |
|---|---|---|---|
| Ethinylestradiol | 0.020 | mg | Active ingredient |
| Drospirenone | 3.0 | mg | Active ingredient |
| Carnauba wax | 3.02 | mg | Protective agent |
| Kollicoat ® IR | 43.96 | mg | Matrix polymer |
| Total | 50 | mg | |

### Example 7E

| Ingredient | Amount | | Function |
|---|---|---|---|
| Ethinylestradiol betadex* | 0.173 | mg | Active ingredient |
| Drospirenone | 3.0 | mg | Active ingredient |
| Carnauba wax | 3.173 | mg | Protective agent |
| Kollicoat ® IR | 43.654 | mg | Matrix polymer |
| Total | 50 | mg | |

*as beta-cyclodextrin clathrate; corresponds to 0.020 mg ethinylestradiol

### Example 7F

| Ingredient | Amount | | Function |
|---|---|---|---|
| Ethinylestradiol | 0.020 | mg | Active ingredient |
| Drospirenone | 3.0 | mg | Active ingredient |
| Carnauba wax | 3.02 | mg | Protective agent |
| Kollicoat ® IR | 42.96 | mg | Matrix polymer |
| Menthol | 1.0 | mg | Taste modifier |
| Total | 50 | mg | |

### Example 7G

| Ingredient | Amount | | Function |
|---|---|---|---|
| Ethinylestradiol betadex* | 0.173 | mg | Active ingredient |
| Drospirenone | 3.0 | mg | Active ingredient |
| Carnauba wax | 3.173 | mg | Protective agent |
| Kollicoat ® IR | 42.654 | mg | Matrix polymer |
| Menthol | 1.0 | mg | Taste modifier |
| Total | 50 | mg | |

*as beta-cyclodextrin clathrate; corresponds to 0.020 mg ethinylestradiol

### Example 7H

| Ingredient | Amount | | Function |
|---|---|---|---|
| Ethinylestradiol | 0.015 | mg | Active ingredient |
| (unprotected) | | | |
| Drospirenone | 3.0 | mg | Active ingredient |
| Eudragit ® E 100 | 12.0 | mg | Protective agent |
| HPMC | 27.485 | mg | Matrix polymer |
| Propylene glycol | 3.75 | mg | Softening agent |
| Sorbitol | 3.75 | mg | Sweetener |
| Total | 50 | mg | |

### Example 7I

| Ingredient | Amount | Function |
|---|---|---|
| Ethinylestradiol | 0.015 mg | Active ingredient |
| (unprotected) | | |
| Drospirenone | 3.0 mg | Active ingredient |
| Eudragit ® E 100 | 12.0 mg | Protective agent |
| HPMC | 34.985 mg | Matrix polymer |
| Total | 50 mg | |

### Example 7J

| Ingredient | Amount | Function |
|---|---|---|
| Ethinylestradiol | 0.015 mg | Active ingredient |
| (unprotected) | | |
| Drospirenone | 3.0 mg | Active ingredient |
| Eudragit ® E 100 | 12.0 mg | Protective agent |
| Kollicoat ® IR | 34.985 mg | Matrix polymer |
| Total | 50 mg | |

### Example 7K

| Ingredient | Amount | Function |
|---|---|---|
| Ethinylestradiol | 0.015 mg | Active ingredient |
| (unprotected) | | |
| Drospirenone | 3.0 mg | Active ingredient |
| Carnauba wax | 3.0 mg | Protective agent |
| Kollicoat ® IR | 43.985 mg | Matrix polymer |
| Total | 50 mg | |

### Example 7L

| Ingredient | Amount | Function |
|---|---|---|
| Ethinylestradiol betadex* | 0.130 mg | Active ingredient |
| (unprotected) | | |
| Drospirenone | 3.0 mg | Active ingredient |
| Carnauba wax | 3.0 mg | Protective agent |
| Kollicoat ® IR | 43.87 mg | Matrix polymer |
| Total | 50 mg | |

*as beta-cyclodextrin clathrate; corresponds to 0.015 mg ethinylestradiol

### Example 7M

| Ingredient | Amount | Function |
|---|---|---|
| Estradiol hemihydrate* | 0.093 mg | Active ingredient |
| (unprotected) | | |
| Drospirenone | 3.0 mg | Active ingredient |
| Eudragit ® E 100 | 12.0 mg | Protective agent |
| HPMC | 27.407 mg | Matrix polymer |
| Propylene glycol | 3.75 mg | Softening agent |
| Sorbitol | 3.75 mg | Sweetener |
| Total | 50 mg | |

*Corresponds to 0.090 mg estradiol

### Example 7N

| Ingredient | Amount | Function |
|---|---|---|
| Estradiol hemihydrate* | 0.093 mg | Active ingredient |
| (unprotected) | | |
| Drospirenone | 3.0 mg | Active ingredient |
| Eudragit ® E 100 | 12.0 mg | Protective agent |
| HPMC | 34.907 mg | Matrix polymer |
| Total | 50 mg | |

*Corresponds to 0.090 mg estradiol

### Example 7O

| Ingredient | Amount | Function |
|---|---|---|
| Estradiol hemihydrate* | 0.093 mg | Active ingredient |
| (unprotected) | | |
| Drospirenone | 3.0 mg | Active ingredient |
| Eudragit ® E 100 | 12.0 mg | Protective agent |
| Kollicoat ® IR | 34.907 mg | Matrix polymer |
| Total | 50 mg | |

*Corresponds to 0.090 mg estradiol

### Example 7P

| Ingredient | Amount | Function |
|---|---|---|
| Estradiol hemihydrate* | 0.093 mg | Active ingredient |
| (unprotected) | | |
| Drospirenone | 3.0 mg | Active ingredient |
| Carnauba wax | 3.0 mg | Protective agent |
| Kollicoat ® IR | 43.907 mg | Matrix polymer |
| Total | 50 mg | |

*Corresponds to 0.090 mg estradiol

### Example 7Q

| Ingredient | Amount | Function |
|---|---|---|
| Estradiol valerate* | 0.118 mg | Active ingredient |
| (unprotected) | | |
| Drospirenone | 3.0 mg | Active ingredient |
| Eudragit ® E 100 | 12.0 mg | Protective agent |
| HPMC | 27.382 mg | Matrix polymer |
| Propylene glycol | 3.75 mg | Softening agent |
| Sorbitol | 3.75 mg | Sweetener |
| Total | 50 mg | |

*Corresponds to 0.090 mg estradiol

### Example 7R

| Ingredient | Amount | Function |
|---|---|---|
| Estradiol valerate* | 0.118 mg | Active ingredient |
| (unprotected) | | |
| Drospirenone | 3.0 mg | Active ingredient |
| Eudragit ® E 100 | 12.0 mg | Protective agent |
| HPMC | 34.882 mg | Matrix polymer |
| Total | 50 mg | |

*Corresponds to 0.090 mg estradiol

### Example 7S

| Ingredient | Amount | Function |
|---|---|---|
| Estradiol valerate* | 0.118 mg | Active ingredient |
| (unprotected) | | |
| Drospirenone | 3.0 mg | Active ingredient |
| Eudragit ® E 100 | 12.0 mg | Protective agent |
| Kollicoat ® IR | 34.882 mg | Matrix polymer |
| Total | 50 mg | |

*Corresponds to 0.090 mg estradiol

### Example 7T

| Ingredient | Amount | Function |
|---|---|---|
| Estradiol valerate* | 0.118 mg | Active ingredient |
| (unprotected) | | |
| Drospirenone | 3.0 mg | Active ingredient |
| Carnauba wax | 3.0 mg | Protective agent |
| Kollicoat ® IR | 43.882 mg | Matrix polymer |
| Total | 50 mg | |

*Corresponds to 0.090 mg estradiol

### Example 7U

| Ingredient | Amount | Function |
|---|---|---|
| Ethinylestradiol | 0.020 mg | Active ingredient |
| Drospirenone | 3.0 mg | Active ingredient |
| Carnauba wax | 3.02 mg | Protective agent |
| HPMC | 43.96 mg | Matrix polymer |
| Total | 50 mg | |

### Example 7V

| Ingredient | Amount | Function |
|---|---|---|
| Ethinylestradiol | 0.020 mg | Active ingredient |
| (unprotected) | | |
| Drospirenone | 3.0 mg | Active ingredient |
| Carnauba wax | 3.0 mg | Protective agent |
| HPMC | 43.98 mg | Matrix polymer |
| Total | 50 mg | |

### Example 7W

| Ingredient | Amount | Function |
|---|---|---|
| Ethinylestradiol | 0.020 mg | Active ingredient |
| Drospirenone | 3.0 mg | Active ingredient |
| Eudragit ® E 100 | 12.18 mg | Protective agent |
| HPMC | 31.05 mg | Matrix polymer |
| Propylene glycol | 3.75 mg | Softening agent |
| Total | 50 mg | |

### Example 7X

| Ingredient | Amount | Function |
|---|---|---|
| Ethinylestradiol | 0.015 mg | Active ingredient |
| (unprotected) | | |
| Drospirenone | 3.0 mg | Active ingredient |
| Eudragit ® E 100 | 12.0 mg | Protective agent |
| HPMC | 31.235 mg | Matrix polymer |
| Propylene glycol | 3.75 mg | Softening agent |
| Total | 50 mg | |

### Example 7Y

| Ingredient | Amount | Function |
|---|---|---|
| Estradiol hemihydrate* | 0.093 mg | Active ingredient |
| (unprotected) | | |
| Drospirenone | 3.0 mg | Active ingredient |
| Eudragit ® E 100 | 12.0 mg | Protective agent |
| HPMC | 31.157 mg | Matrix polymer |
| Propylene glycol | 3.75 mg | Softening agent |
| Total | 50 mg | |

*Corresponds to 0.090 mg estradiol

### Example 7Z

| Ingredient | Amount | Function |
|---|---|---|
| Estradiol valerate* | 0.118 mg | Active ingredient |
| (unprotected) | | |
| Drospirenone | 3.0 mg | Active ingredient |
| Eudragit ® E 100 | 12.0 mg | Protective agent |
| HPMC | 31.132 mg | Matrix polymer |
| Propylene glycol | 3.75 mg | Softening agent |
| Total | 50 mg | |

*Corresponds to 0.090 mg estradiol

| Ingredient | Amount | Function |
|---|---|---|
| Drospirenone | 3.0 mg | Active ingredient |
| Eudragit ® E 100 | 12.0 mg | Protective agent |
| HPMC | 27.5 mg | Matrix polymer |
| Propylene glycol | 3.75 mg | Softening agent |
| Sorbitol | 3.75 mg | Sweetener |
| Total | 50 mg | |

### Example 7AB

| Ingredient | Amount | Function |
|---|---|---|
| Drospirenone | 3.0 mg | Active ingredient |
| Eudragit ® E 100 | 12.0 mg | Protective agent |
| HPMC | 31.25 mg | Matrix polymer |
| Propylene glycol | 3.75 mg | Softening agent |
| Total | 50 mg | |

### Example 7AC

| Ingredient | Amount | Function |
|---|---|---|
| Drospirenone | 3.0 mg | Active ingredient |
| Eudragit ® E 100 | 12.0 mg | Protective agent |
| Kollicoat ® IR | 35.0 mg | Matrix polymer |
| Total | 50 mg | |

### Example 7AD

| Ingredient | Amount | Function |
|---|---|---|
| Drospirenone | 3.0 mg | Active ingredient |
| Carnauba wax | 3.0 mg | Protective agent |
| Kollicoat ® IR | 44.0 mg | Matrix polymer |
| Total | 50 mg | |

### Example 7AF

| Ingredient | Amount | Function |
|---|---|---|
| Drospirenone | 3.0 mg | Active ingredient |
| Carnauba wax | 3.0 mg | Protective agent |
| HPMC | 44.0 mg | Matrix polymer |
| Total | 50 mg | |

### Example 7AF

| Ingredient | Amount | Function |
|---|---|---|
| Ethinylestradiol | 0.030 mg | Active ingredient |
| Dienogest | 2.0 mg | Active ingredient |
| Carnauba wax | 2.03 mg | Protective agent |
| Kollicoat ® IR | 30.94 mg | Matrix polymer |
| Total | 35 mg | |

### Example 7AG

| Ingredient | Amount | Function |
|---|---|---|
| Ethinylestradiol | 0.030 mg | Active ingredient |
| Dienogest | 2.0 mg | Active ingredient |
| Eudragit ® E 100 | 8.27 mg | Protective agent |
| HPMC | 35.95 mg | Matrix polymer |
| Propylene glycol | 3.75 mg | Softening agent |
| Total | 50 mg | |

### Example 7AH

| Ingredient | Amount | Function |
|---|---|---|
| Ethinylestradial | 0.015 mg | Active ingredient |
| (unprotected) | | |
| Dienogest | 2.0 mg | Active ingredient |
| Carnauba wax | 2.00 mg | Protective agent |
| Kollicoat ® IR | 30.985 mg | Matrix polymer |
| Total | 35 mg | |

### Example 7AI

| Ingredient | Amount | Function |
|---|---|---|
| Ethinylestradiol | 0.015 mg | Active ingredient |
| (unprotected) | | |
| Dienogest | 2.0 mg | Active ingredient |
| Eudragit ® E 100 | 8.00 mg | Protective agent |
| HPMC | 36.235 mg | Matrix polymer |
| Propylene glycol | 3.75 mg | Softening agent |
| Total | 50 mg | |

### Example 7AJ

| Ingredient | Amount | Function |
|---|---|---|
| Dienogest | 2.0 mg | Active ingredient |
| Carnauba wax | 2.00 mg | Protective agent |
| Kollicoat ® IR | 31.00 mg | Matrix polymer |
| Total | 35 mg | |

### Example 7AK

| Ingredient | Amount | Function |
|---|---|---|
| Dienogest | 2.0 mg | Active ingredient |
| Eudragit ® E 100 | 8.00 mg | Protective agent |
| HPMC | 36.25 mg | Matrix polymer |
| Propylene glycol | 3.75 mg | Softening agent |
| Total | 50 mg | |

The 50 mg and 35 mg wafers described in this example have a surface area of 7 cm² and 5 cm², respectively. Also, wafers similar to the 50 mg wafers described above, but having a total weight of 40 mg or 45 mg, can be prepared analogously by using a corresponding lower amount of the matrix polymer. As will be understood, the amount of therapeutically active agent will be the same independently of the total weight and the surface dimension of the wafer.

Likewise, wafers similar to those described in examples 7A to 7AK above, but containing 2 mg dienogest, 0.06 mg gestodene or 0.075 mg gestodene instead of 3 mg drospirenone, can be prepared analogously by using a corresponding higher amount of the matrix polymer.

### Example 8A

### In Vitro Dissolution Test Representing the Conditions in the Mouth

The dosage form is placed onto the bottom of a 100 ml glass beaker. Then, 10.0 ml of simulated saliva pH 6.0 (composition: 1.436 g disodium phosphate dihydrate, 7.98 g monopotassium phosphate, and 8.0 g sodium chloride are dissolved in 950 ml water, adjusted to pH 6.0 and made up to 1000 ml) at 37° C. is added into the beaker (dissolution medium). The experiment is performed without any stirring or shaking, except for a gentle shaking within the first five seconds of the experiment in order to safeguard complete wetting of the dosage form. After 3 minutes, the content of the beaker is inspected visually, and a sample of the liquid is drawn, filtered (Spartan 30B filter) and analyzed for the content of the drospirenone.

Wafers prepared from the coating solution described in examples 2A and manufactured as described in example 3A were subjected to the above in vitro dissolution test representing the conditions in the mouth. The experiment was performed in triplicate. All wafers were completely disintegrated after 3 minutes. The individual amounts of drospirenone released after 3 minutes were 3.5%, 2.8%, and 3.5%, respectively (mean 3.3%).

Wafers prepared from the coating solution described in examples 21 and manufactured as described in example 3A were subjected to the above in vitro dissolution test representing the conditions in the mouth. The experiment was performed in triplicate. All wafers were completely disintegrated after 3 minutes. The individual amounts of drospirenone released after 3 minutes were 21.2%, 20.4%, and 12.5%, respectively (mean 18.0%).

### Example 8B

### In Vitro Dissolution Test Representing the Conditions in the Intestine

The release of the drug substance(s) is investigated by the USP XXXI Paddle Method (apparatus 2) using 1000 ml of 0.05M phosphate buffer pH 6.0 with 0.5% (w/v) sodium dodecyl sulphate at 37° C. as dissolution medium and 50 rpm as the stirring rate.

Wafers prepared from the coating solution described in examples 2A and manufactured as described in example 3A were subjected to the above in vitro dissolution test representing the conditions in the intestine. It was found, that about 75% of the drospirenone was dissolved after 15 minutes, and about 80% of the drospirenone was dissolved after 30 minutes.

Wafers prepared from the coating solution described in examples 2I and manufactured as described in example 3A have been subjected to the above in vitro dissolution test representing the conditions in the intestine. It was found, that about 95% of the drospirenone was dissolved after 15 minutes.

### Example 8C

### In Vitro Dissolution Test Representing the Conditions in the Gastro-Intestinal Tract

The release of the drug substance(s) is investigated by the USP XXXI Paddle

Method (apparatus 2) using 1000 ml of 0.05 M acetate buffer pH 4.5 with 0.5% (w/v) sodium dodecyl sulphate at 37° C. as dissolution medium and 50 rpm as the stirring rate.

Wafers according to examples 7D, 7K, 7P, and manufactured as described in example 3b have been subjected to the above in vitro dissolution test representing the conditions in the gastro-intestinal tract. It was found, that about 95% of the drospirenone was dissolved after 15 minutes.

### Example 8D

### In Vitro Dissolution Test Representing the Conditions in the Gastro-Intestinal Tract

The release of the drug substance(s) is investigated by the USP XXXI Paddle Method (apparatus 2) using 1000 ml of 0.05 M acetate buffer pH 4.5 at 37° C. as dissolution medium and 50 rpm as the stirring rate.

Wafers according to 7W, 7X, 7Y, and manufactured as described in example 3b have been subjected to the above in vitro dissolution test representing the conditions in the gastro-intestinal tract. It was found, that about 90% of the drospirenone was dissolved after 15 minutes

### Example 9

### Content Uniformity

Wafers according to examples 7A, 7D, 7K, 7P, 7X and manufactured as described in example 3b have been subjected to the content uniformity test according to the United States Pharmacopoeia (USP). The assay was determined via HPLC. The below acceptance values were found.

| | | Acceptance |
|---|---|---|
| Example | Therapeutically active agent | value |
| 7A | Drospirenone protected | 9.8% |
| | Ethinylestradiol protected | 9.2% |
| 7D | Drospirenone protected | 6.6% |
| | Ethinylestradiol protected | 5.8% |
| 7K | Drospirenone protected | 1.9% |
| | Ethinylestradiol unprotected | 6.9% |
| 7P | Drospirenone protected | 2.4% |
| | Estradiol hemihydrate unprotected | 10.9% |
| 7X | Drospirenone protected | 10.5% |
| | Ethinylestradiol unprotected | 10.9% |

1. A unit dosage form comprising a thin water-soluble film matrix, wherein
a) said film matrix comprises at least one water-soluble matrix polymer;
b) said film matrix comprises particles where said particles comprises at least one progestin and at least one protective agent, and where said particles have a d₉₀ particle size of ≦280 μm; and
c) said film matrix has a thickness of 300 μm.

2. The unit dosage form according claim 1, wherein said progestin is embedded in said protective agent.

3. The unit dosage form according to claim 2, wherein said progestin is present in a solid dispersion in said protective agent.

4. The unit dosage form according to claim 1, wherein said progestin is coated with said protective agent.

5. The unit dosage form according to claim 1, wherein said protective agent is a cationic polymethacrylate.

6. The unit dosage form according to claim 1, wherein said protective agent is a wax.

7. The unit dosage form according to claim 6, wherein said wax is carnauba wax.

8. The unit dosage form according to claim 1, wherein said particles have a d₉₀ particle size of 5.250 μm, such as a d₉₀ particle size of ≦200 μm, preferably a d₉₀ particle size of 5.175 μm, such as a d₉₀ particle size of ≦150 μm, e.g. a d₉₀ particle size of ≦100 μm.

9. The unit dosage form according to claim 1, wherein said particles have a d₉₀ particle size in the range of from 30-280 μm, such as in the range of from 40-250 μm, e.g. in the range of from 50-200 μm or in the range of from 50-150 μm.

10. The unit dosage form according to claim 1, wherein said progestin is selected from the group consisting of levo-norgestrel, norgestrel, norethindrone (norethisterone), dienogest, norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desogestrel, 3-keto-desogestrel, norgestimate, gestodene, tibolone, cyproterone acetate, dienogest and drospirenone.

11. The unit dosage form according to claim 10, wherein said progestin is selected from the group consisting of gestodene, dienogest and drospirenone.

12. The unit dosage form according to claim 11, wherein said unit dosage form comprises 0.25-5 mg drospirenone, such as 1-4 mg drospirenone, e.g. 2-4 mg drospirenone, preferably 2.5-3.5 mg drospirenone, most preferably about 3 mg drospirenone.

13. The unit dosage form according to claim 1, wherein said water-soluble matrix polymer is selected from the group consisting of a cellulosic material, a gum, a protein, a starch, a synthetic polymer, a glucan, and mixtures thereof.

14. The unit dosage form according to claim 1, wherein said film matrix has a thickness of ≦250 μm, preferably ≦200 μm, such as ≦150 μm, more preferably ≦120, such as ≦100 μm.

15. The unit dosage form according to claim 14, wherein said film matrix has a thickness in the range of from 10-150 μm, such as 20-125 μm, e.g. 30-100 μm, preferably 35-90 μm, more preferably 40-80 μm.

16. The unit dosage form according to claim 1, wherein said unit dosage form further comprises at least one estrogen.

17. The unit dosage form according to claim 16, wherein
a) said film matrix comprises at least one water-soluble matrix polymer;
b) said film matrix comprises particles where said particles comprises at least one progestin, at least one estrogen and at least one protective agent, and where said particles have a d₉₀ particle size of ≦280 μm; and
c) said film matrix has a thickness of ≦300 μm.

18. The unit dosage form according to claim 16, wherein
a) said film matrix comprises at least one water-soluble matrix polymer;
b) said film matrix comprises particles where said particles comprises at least one progestin and at least one protective agent, and where said particles have a d₉₀ particle size of ≦280 μm;
c) said film matrix comprises particles where said particles comprises at least one estrogen and at least one protective agent, and where said particles have a d₉₀ particle size of ≦280 μm;
d) said film matrix has a thickness of ≦300 μm.

19. The unit dosage form according to claim 16, wherein said film matrix comprises at least one surfactant.

20. The unit dosage form according to claim 16, wherein
a) said film matrix comprises at least one water-soluble matrix polymer, wherein at least one estrogen is dispersed in said water-soluble matrix polymer;
b) said film matrix comprises particles where said particles comprises at least one progestin and at least one protective agent, and where said particles have a d₉₀ particle size of ≦280 μm; and
c) said film matrix has a thickness of 300 μm.

21. The unit dosage form according to claim 16, wherein said estrogen is selected from the group consisting of ethinylestradiol, estradiol including therapeutically acceptable derivates of estradiol, estrone, mestranol, estriol, estriol succinate and conjugated estrogens.

22. The unit dosage form according to claim 16, wherein less than 25% (w/w), preferably less than 20% (w/w), more preferably less than 15% (w/w), most preferably less than 5% (w/w) of the progestin is dissolved from the unit dosage form within 3 minutes when the unit dosage form is placed into a beaker with 10 ml of simulated saliva pH 6.0 at 37° C. as dissolution medium.

23. The unit dosage form according to claim 16 for use as a medicament.

24. A unit dosage form according to claim 16 for the inhibition of ovulation in a female mammal.

25. A unit dosage form according to claim 16 for providing contraception in a female mammal.
